(19) United States
(12) Patent Application Publication
(10) Pub. No.: US 2012/0010161 A1
(43) Pub. Date: Jan. 12, 2012

(54) NOVEL AZAPEPTIDE OR AZAPEPTIDOMIMETIC COMPOUNDS INHIBITING BCRP AND/OR P-GP

(75) Inventor: Joelle Paris, Lyon (FR)

(73) Assignees: CENTRE NATIONAL DE LA RECHERCHE SCIENTIFIQUE, PARIS CEDIX 16 (FR); UNIVERSITE CLAUDE BERNARD LYON I, VILLEURBANNE CEDEX (FR)

(21) Appl. No.: 13/145,657

(22) PCT Filed: Jan. 25, 2010
(86) PCT No.: PCT/FR2010/050107

§ 371 Date: Sep. 29, 2011

(30) Foreign Application Priority Data
Jan. 26, 2009 (FR) 0950450

(51) Int. Cl. A61K 31/7048; A61K 31/7072; A61K 31/5377; A61K 31/519; A61K 31/506; A61K 31/496; A61K 31/52; A61K 31/513; A61K 31/4375; A61K 31/445; A61K 31/4184; A61K 31/351; C07D 211/00; C07C 229/00; C07C 229/34; A61P 43/00; A61K 31/704

(52) U.S. Cl. 514/30; 514/34; 514/50; 514/234.5; 514/249; 514/252.18; 514/254.07; 514/263.4; 514/274; 514/283; 514/315; 514/394; 514/459; 546/246; 562/443; 562/561

(57) ABSTRACT

The present invention relates to compounds of azapeptide or azapeptidomimetic type of formula (I):

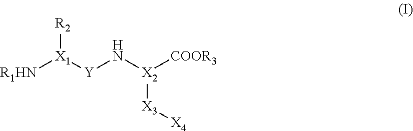

in which R₁, R₂, R₃, X₁, X₂, X₃, X₄ and Y are as defined in claim 1, to pharmaceutical compositions containing them and to such compounds as adjuvant for an anticancer or anti-infectious medicament.

The field of the invention is generally that of therapeutic chemistry, more particularly applied to improving the efficacy of chemotherapeutic treatments, and especially cancerology treatments. More specifically, the subject of the present invention is compounds of azapeptide or azapeptidomimetic type that behave as inhibitors of certain efflux proteins such as BCRP (breast cancer resistance protein, also known as ABCG2), and also P-gp (pleiotropic glycoprotein, also known as ABCB1).

A major problem that confronts the chemotherapeutic treatment of cancers and infections of viral, bacterial, fungal or parasitic origin is the intrinsic or acquired resistance of malignant cells or of bacteria, fungi, viruses or parasites. Among the phenomena of acquired resistance is multidrug resistance, which is reflected by a decrease in the intracellular concentration of the chemotherapeutic agent due to the overexpression of transport proteins of ABC type (ATP-binding cassette) which expel the chemotherapeutic agent from the target tumor cells or cells infected with an infectious agent (virus, bacterium, fungus, parasite, etc.). These proteins are also expressed physiologically, especially in the gastrointestinal tract and in the blood-brain barrier.

The notion of multidrug resistance arises from the fact that the cells become resistant not only to the chemotherapeutic agents administered, but also to a large number of structurally unrelated molecules. This elimination arises mainly via the action of efflux pumps, of which three main ones are identified in man. These efflux pumps are ABCB1, ABCC1 and ABCG2, also known, respectively, as P-gp, discovered by Juliano and Ling in 1976 (Biochim. Biophys. Acta, 1976, 455, 152-162), MRP1 (multidrug resistance protein 1), discovered by Cole and Deeley 16 years later in lung cancer (Science, 1992, 258, 1650-1654) and BCRP or MXR (mitoxantrone resistance) discovered more recently by Ross et al., on the one hand (1 Natl Cancer Inst., 1999, 91, 429-433) and by Bates et al., on the other hand (J. Cell Sci., 2000, 113 (Pt 11), 2011-2021). These efflux proteins are inserted into the plasma membrane via a transmembrane domain linked to a large intracellular domain. The latter is formed from two nucleotide binding sites (Nucleotide-Binding Domain, NBD). A small domain is present on the extracellular face of these proteins. The structure of these proteins is probably similar to that of the two prokaryotic efflux proteins Sav1866 (Dawson & Locher, Nature 2006 443 (7108) 180-5) and MsbA (Ward et al., Proc. Natl. Acad. Sci. USA 2007, 104 (48) 19005-10). They permit ATP-dependent efflux of varied chemotherapeutic agents out of the cell.

Modulation of the activity of these transport proteins may make it possible to restore a concentration level that is efficient as a medicament and to limit chemotherapy failures due to the MDR phenomenon. In this context, numerous studies have been performed to obtain inhibitors that block the efflux function of these proteins; agents with this type of activity are known as chemosensitizers. However, none of them has satisfied the criteria of efficacy, nontoxicity and selectivity, the latter being directed toward avoiding disrupting the physiological functions of the other transport proteins. The majority are also competitive inhibitors, i.e. they come into competition with the substrate at the site of transport; they are thus pseudosubstrates, which, as a result, may become the substrate of a transporter of the same type, due to the capacity for adaptation of these transporters to different substrates. In this context, it is thus important to select noncompetitive inhibitors.

These efflux proteins, which were initially discovered in the course of resistance to anticancer agents, have also recently been associated with the development of resistance to antiviral treatments. Thus, ABCB1 modulates the oral bioavailability of protease inhibitors and their penetration into the central nervous system (Kim, Top HIV Med., 2003, 11, 136-139); similarly, ABCG2 effluxes zidovudine/AZT, lamivudine/Epivir and abacavir/Ziagen (Wang et al., Mol. Pharmacol. 2003, 63, 65-72; Weiss et al., J. Antimicrob. Chemother., 2007 dkl474.). Their transport spectrum is very broad, which makes them capable of effluxing a large number of compounds, of varied chemical structure, and used, for example, for treating the various pathologies, including cancer.

These efflux proteins are naturally expressed in normal tissues so as to expel the toxins and waste produced by the cells (e.g. lung, kidney and liver). They are also produced in the blood-brain barrier to prevent toxins from entering the brain. One of the potential strategies for inversing the MDR phenotype consists in coadministering the chemotherapeutic agent and an efflux protein inhibitor. Several molecules have been developed for this purpose. The three generations of inhibitors developed for blocking the efflux activity of these proteins are presented in Table 1.

Table 1a and 1b: the symbols indicate either that the inhibitor is active (+) or that it is inactive (−) on a specific transporter, or that the effect is unknown (?). Taken from Current Opinion in Pharmacology, 2006, 6, 350-354.

TABLE 1a

Second-generation inhibitors

| | PSC833 | VX-710 |
|---|---|---|
| Transporter | (valspodar) | (biricodar) |
| ABCB1 | + | + |
| ABCC1 | + | + |
| ABCG2 | − | + |

TABLE 1b

| | Third-generation inhibitors | | | | |
|---|---|---|---|---|---|
| | GF 120918 | R101933 | XR9576 | Ly335979 | ONT-093 |
| Transporter | (elacridar) | (laniquidar) | (tariquidar) | (zosuquidar) | (ontogen) |
| ABCB1 | + | + | + | + | + |
| ABCC1 | − | ? | − | − | − |
| ABCG2 | + | ? | + | − | ? |

Certain inhibitors, such as tariquidar, interact with several proteins. In particular, they significantly inhibit, in addition to ABCB1 and/or ABCG2, ABCC1. This is not necessarily an advantage, since, ideally, a reversing agent should target only one efflux protein, so as to preserve the physiological functions of the others in healthy cells. Moreover, other agents, such as zosuquidar, have recently been stopped at phase III on account of adverse effects. These examples demonstrate that it is difficult to find novel types of reversible agent that are both powerful and specific, and that have a minimum level of adverse effects.

The present invention proposes to provide novel compounds of the azapeptide or azapeptidomimetic type that behave as ABCG2 and/or ABCB1 inhibitors. Furthermore, another object of the invention is to provide ABCG2 and/or ABCB1 inhibitors that do not show significant ABCC1 inhibition. Some of the compounds of the invention also show high affinity and, frequently, selectivity for ABCG2 or ABCB1 and/or behave as noncompetitive inhibitors.

Another object of the invention is to propose novel compounds that are noncytotoxic.

Another object of the invention is to propose novel compounds that are relatively easy to prepare and of reasonable cost price.

In this context, one subject of the present invention is compounds of azapeptide or azapeptidomimetic type of formula (I):

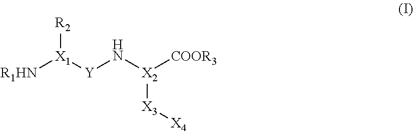

in which:
R₁ represents a protecting group for an amino group, preferably a group —C(O)OR′₁ with R′₁ which represents an alkyl group of 1 to 12 carbon atoms or a group —(CH₂)ₘ₁R″₁ with R″₁ which represents an aryl group (for example phenyl), cycloalkyl or fluorenyl and m1 which is equal to 0, 1, 2 or 3,
X₁ and X₂ are identical or different and represent —N— or —CH—, it being understood that at least one represents —N—,
R₂ represents a side chain optionally in protected form of an amino acid or an amino acid analog, or an optionally substituted aryl group (for example phenyl) or an optionally substituted heteroaryl group,
Y represents —CH₂— or —C(O)—,
—X₃-X₄ represents either a group —(CH₂)ₙ—NHR₄ with n equal to 3, 4, 5 or 6, or a group chosen from:

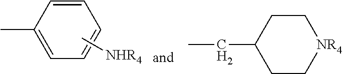

with R₄ which represents a protecting group for an amino group, preferably a group —C(O)OR′₄ with R^{′}₄ which represents an alkyl group of 1 to 12 carbon atoms or a group —(CH₂)ₘ₄R″₄ with R^{″}₄ which represents an aryl group (for example phenyl) or cycloalkyl and m4 which is equal to 0, 1, 2 or 3,
   R₃ represents an alkyl group of 1 to 12 carbon atoms, or a group —(CH₂)ₘ₃R″₃ with R″₃ which represents a cycloalkyl or aryl group (for example phenyl), said cycloalkyl and aryl groups possibly being unsubstituted or substituted with one or more groups chosen from:
   —CH₃, —CF₃, —COOH, —NO₂, —Cl and NH₂ and m3 which is equal to 0, 1, 2 or 3,
in the form of a pure optical isomer or a mixture of optical isomers, and also the salts, solvates or hydrates thereof.

According to one embodiment variant, the compounds of formula (I) as defined previously have as substituent R₂ a group -L-COOR′₂ with L which represents an aryl group, preferably phenyl, or a chain —(CH₂)ₘ₂— with m₂ which is equal to 1, 2 or 3 and R′₂ which represents an alkyl group of 1 to 12 carbon atoms or a group —(CH₂)_{m′2}R″₂ with R″₂ which represents an optionally substituted aryl (for example phenyl) or cycloalkyl group, and m′2 which is equal to 0, 1, 2 or 3.

One subgroup of compounds according to the invention is composed of the compounds of formula (IA), in the form of a pure isomer or a mixture of isomers, and also the salts, solvates or hydrates thereof:

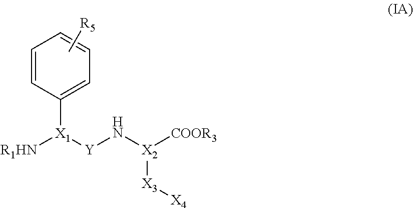

in which R₅ represents a group —NH₂, —NO₂, —OH, —O-alkyl of 1 to 8 carbon atoms, O—(CH₂)ₘ₅R′₅ with R′₅ which represents an aryl group (for example phenyl) and m5 which is equal to 0 or 1 or alternatively R₅ represents a group —COOR″₅ with R″₅ which represents an alkyl group of 1 to 12 carbon atoms or a group —(CH₂)_{m′5}R′″₅ with R′″₅ which represents an aryl group (for example phenyl) or cycloalkyl and m′5 which is equal to 0, 1, 2 or 3, R₁, X₁, Y, X₂, X₃, X₄ and R₃ being as defined for (I).

Advantageously, the compounds of formula (IA) have one or other of the characteristics below or a combination of some or all of these characteristics, when they are not mutually exclusive:
R₅ represents a group —COOR″₅ with R″₅ which represents an ethyl, benzyl or —CH[CH(CH₃)₂]₂ group,
R₅ is in the meta position, and in this case selectivity toward BCRP is preferred,
the group R₅ is in the para position, and in this case selectivity toward P-gp is preferred,
X₁ represents —N— and X₂ represents —CH—,
X₁ and X₂ represent N,
Y represents —C(O)—,
—X₃-X₄ represents a group —(CH₂)ₙ—NHR₄ with n equal to 4 or 6 and R₄ as defined for (I).

A subgroup of compounds of the invention is composed of the compounds of formula (IB), in the form of a pure isomer or a mixture of isomers, and also the salts, solvates or hydrates thereof:

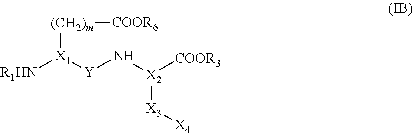

in which m is equal to 1, 2 or 3, R₆ represents an alkyl group of 1 to 12 carbon atoms, or a group —(CH₂)ₘ₆R′₆ with R′₆ which represents a cycloalkyl or aryl group (for example phenyl), said cycloalkyl and aryl groups (for example phenyl) possibly being unsubstituted or substituted with one or more groups chosen from: —CH₃, —CF₃, —COOH, —NO₂, —Cl and NH₂ and m6 which is equal to 0, 1, 2 or 3, R₁, X₁, Y, X₂, X₃, X₄ and R₃ being as defined for (I)

Advantageously, the compounds of formula (IB) have one or other of the characteristics below or a combination of some or all of these characteristics, when they are not mutually exclusive:
m is equal to 1 or 2,
R₆ represents an ethyl, benzyl or —CH[CH(CH₃)₂]₂ group,
X₁ represents —CH— and X₂ represents —N—,
X₁ represents —N— and X₂ represents —CH—,
—X₃-X₄ represents a group —(CH₂)ₙ—NHR₄ with n equal to 6 and R₄ as defined for (I).

Also preferred are the compounds of formula (I), (IA) or (IB) which have one, two or three of the following characteristics:
R₁ represents a group —COOR′₁ and R′₁ represents a tert-butyl or benzyl group,
R₃ represents a tert-butyl or benzyl group,
R₄ represents —C(O)OR′₄ with R′₄ which represents a benzyl or tert-butyl group.

As specific examples of compounds according to the invention, mention may be made of compounds of azapeptide or azapeptidomimetic type chosen from:
tert-butyl ester of (2S)-6-(benzyloxycarbonylamino)-2-[1-(2′-(benzyloxycarbonyl)phenyl)-2-(tert-butoxycarbonyl)hydrazinocarbonylamino]-hexanoic acid (IA.1)
tert-butyl ester of (2S)-6-(benzyloxycarbonylamino)-2-[1-(3′-(benzyloxycarbonyl)phenyl)-2-(tert-butoxycarbonyl)hydrazinocarbonylamino]-hexanoic acid (IA.2)
tert-butyl ester of (2S)-6-(benzyloxycarbonylamino)-2-[1-(4′-(benzyloxycarbonyl)phenyl)-2-(tert-butoxycarbonyl)hydrazinocarbonylamino]-hexanoic acid (IA.3)
benzyl ester of (2R)-6-(benzyloxycarbonylamino)-2-[1-(3′-(benzyloxycarbonyl)phenyl)-2-(tert-butoxycarbonyl)hydrazinocarbonylamino]-hexanoic acid (IA.4)
benzyl ester of (2S)-6-(benzyloxycarbonylamino)-2-[1-(3′-(benzyloxycarbonyl)phenyl)-2-(tert-butoxycarbonyl)hydrazinocarbonylamino]-hexanoic acid (IA.5)
tert-butyl ester of (2S)-6-(benzyloxycarbonylamino)-2-[1-(3′-(phenyloxycarbonyl)phenyl)-2-(tert-butoxycarbonyl)hydrazinocarbonyl-amino]hexanoic acid (IA.6)
benzyl ester of 2-[1-(3′-(benzyloxycarbonyl)phenyl)-2-(tert-butoxycarbonyl)hydrazinocarbonyl]-1-(6-benzyloxycarbonyl-aminohexyl)hydrazinocarboxylic acid (IA.7)
benzyl ester of 2-[1-(3′-(benzyloxycarbonyl)phenyl)-2-(tert-butoxycarbonyl)hydrazinocarbonyl]-1-(6-tert-butyloxycarbonylaminohexyl)-hydrazinocarboxylic acid (IA.8)
benzyl ester of (3S)-4-[2-(6-benzyloxycarbonylaminohexyl)-2-tert-butoxycarbonylhydrazino]-3-tert-butoxycarbonylaminobutyric acid (IB.1)
benzyl ester of (3S)-4-[2-(3-benzyloxycarbonylaminopropyl)-2-tert-butoxycarbonylhydrazino]-3-tert-butoxycarbonylamino-4-oxobutyric acid (IB.2)
benzyl ester of (3S)-4-[2-(4-benzyloxycarbonylaminophenyl)-2-tert-butoxycarbonylhydrazino]-3-tert-butoxycarbonylamino-4-oxobutyric acid (IB.3)
benzyl ester of (4S)-5-[2-(4-benzyloxycarbonylaminophenyl)-2-tert-butoxycarbonylhydrazino]-4-tert-butoxycarbonylamino-5-oxopentanoic acid (IB.4)
benzyl ester of (3S)-4-[2-(5-benzyloxycarbonylaminopentyl)-2-tert-butoxycarbonylhydrazino]-3-tert-butoxycarbonylamino-4-oxobutyric acid (IB.5)
benzyl ester of 4-[2-((2S)-3-benzyloxycarbonyl-2-tertbutoxycarbonyl-aminopropyl)-1-tert-butoxycarbonylhydrazinomethyl]piperidine-1-carboxylic acid (IB.6)
benzyl ester of 4-[2-((2S)-3-benzyloxycarbonyl-2-tert-butoxycarbonyl-aminopropionyl)-1-tert-butoxycarbonylhydrazinomethyl]piperidine-1-carboxylic acid (IB.7)
benzyl ester of (3S)-4-[2-(5-benzyloxycarbonylaminopentyl)-2-tert-butoxycarbonylhydrazino]-3-tert-butoxwarbonylaminobutyric acid (IB.8)
benzyl ester of (3S)-4-[2-(6-benzyloxycarbonylaminohexyl)-2-tert-butoxycarbonylhydrazino]-3-tert-butoxycarbonylamino-4-oxobutyric acid (IB.9)
benzyl ester of (3S)-3-tert-butoxycarbonylamino-4-[2-tert-butoxycarbonyl-2-(6-tert-butoxycarbonylaminohexyl)hydrazino]butyric acid (IB.10)
benzyl ester of (3S)-4-[2-benzyloxycarbonyl-2-(6-benzyloxycarbonyl-aminohexyl)hydrazino]-3-tert-butoxycarbonylaminobutyric acid (IB.11)
benzyl ester of (3S)-3-benzyloxycarbonylamino-4-[2-benzyloxycarbonyl-2-(6-benzyloxycarbonylaminohexyl)hydrazino]butyric acid (IB.12)
benzyl ester of (3S)-4-[2-benzyloxycarbonyl-2-(6-tert-butoxycarbonyl-aminohexyl)hydrazino]-3-tert-butoxycarbonylaminobutyric acid (IB.13)
tert-butyl ester of (2S)-6-(benzyloxycarbonylamino)-2-[1-(benzyloxycarbonylmethyl)-2-(tert-butoxycarbonyl)-hydrazinocarbonylamino]hexanoic acid (IB.14),
   and also salts, solvates and/or hydrates thereof, and especially the pharmaceutically acceptable forms thereof.

The compounds according to the invention, of synthetic azapeptide or azapeptidomimetic type, have inhibitory properties on BCRP/ABCG2 and/or on P-gp/ABCB1. The compounds according to the invention have a low IC₅₀ value, which may be as low as 2 μM, or even 0.3 μM or less, with respect to at least one of these two proteins. The IC₅₀ is defined as the half-concentration of compound that blocks 50% of the efflux of therapeutic agent. The tests used to determine the activity of the compounds are described in detail in the examples in the “biological activity” section. The compounds according to the invention do not show significant inhibition of ABCC1. In particular, most of the compounds according to the invention have a percentage efficacy at 10 μM, with respect to this protein, of less than 15%. Certain compounds, such as compounds (IA.2) and (IA.3), also prove to be selective toward one or other of the proteins BCRP or P-gp. Some, such as compounds (IA.2) and (IB.11) also prove to be noncompetitive.

The compounds according to the invention are hydrophobic azapeptides or azapeptidomimetics in which a nitrogen atom has been introduced in place of the CHα, either into the N-terminal amino acid (N-aza) or into the C-terminal amino acid (C-aza), for the purpose firstly of increasing their stability and bioavailability, and secondly of promoting/stabilizing the active conformation of the molecule that is the most efficient. The compounds according to the invention make it possible to block the efflux function of ABC transporters that is amplified during chemotherapeutic treatments. ABCs modulate, via their transport/efflux/rejection, the pharmacokinetics of medicaments, whether they are anticancer, antiviral, antibacterial, antifungal or antiparasitic, or more broadly used in the context of a given pathology.

Another subject of the invention thus relates to the compounds defined previously as adjuvants for a chemotherapeutic treatment administered to a patient, for which the body of the treated patient develops a resistance, intended to restore the activity of the therapeutic treatment. The compounds according to the invention may especially be used as adjuvant for an anticancer or anti-infectious medicament.

Preferably, the compounds according to the invention may be defined as adjuvants:

for a medicament chosen from anthracyclines, topoisomerase inhibitors, antimetabolic agents (antifolates), tyrosine kinase inhibitors, antiviral agents (reverse transcriptase inhibitors), antiparasitic agents and antifungal agents,

or for a medicament chosen from daunorubicin, doxorubicin, mitoxantrone, camptothecin and derivatives thereof, irinotecan, topotecan, indolocarbazoles, methotrexate, imatinib, gefitinib, zidovudine, lamivudine, abacavir, ivermectin, albendazole, oxfendazole and ketoconazole.

According to another of its aspects, the present invention relates to the use of a compound of formula (I), (IA) or (IB), as defined above, optionally in hydrated or solvated form, or in the form of a pharmaceutically acceptable salt, for the manufacture of a medicament for improving the effect of a chemotherapeutic treatment, especially in cancerology.

The invention relates to a medicament comprising, separately or together, (i) a compound according to the invention and (ii) a chemotherapeutic agent that is effective against cancer or the complaint that it is desired to treat. According to one particular embodiment of the invention, the medicament is in the form of a single pharmaceutical composition combining, in the same formulation, (i) the compound according to the invention and (ii) the chemotherapeutic agent that is effective against cancer or the complaint that it is desired to treat.

Thus, a subject of the invention is also pharmaceutical compositions comprising a compound of formula (I), (IA) or (IB) as defined previously, optionally in the form of a pharmaceutically acceptable salt, solvate or hydrate, for treating a cancer or for an anti-infectious treatment, in combination with a chemotherapeutic agent that is effective against cancer or the complaint that it is desired to treat.

The description below allows a better understanding of the invention. To begin with, a certain number of definitions are recalled.

The term “alkyl group” means a linear or branched saturated hydrocarbon-based chain. As examples of alkyl groups comprising from 1 to 12 carbon atoms and preferably from 1 to 7 carbon atoms, mention may be made of methyl, ethyl, n-propyl, iso-propyl, n-butyl, tert-butyl, sec-butyl, n-pentyl, n-hexyl, n-heptyl and —CH[CH(CH₃)₂]₂ groups.

The term “cycloalkyl group” denotes a saturated cyclic hydrocarbon-based chain comprising from 3 to 7 carbon atoms. Examples of cycloalkyl groups that may be mentioned include cyclopropyl, cyclobutyl, cyclopentyl, cyclohexyl and cycloheptyl groups.

The term “aryl group” denotes a mono-, bi- or polycyclic carbocycle preferably comprising from 6 to 12 carbon atoms, comprising at least one aromatic group, for example a phenyl, cinnamyl or naphthyl radical. Phenyl is the particularly preferred aryl group.

The term “heteroaryl group” means an aryl group interrupted with one or more heteroatoms chosen from nitrogen, oxygen and sulfur atoms, for example chosen from pyridyl, furyl, thienyl, isoxazolyl, oxadiazolyl, oxazolyl, benzimidazole, indolyl and benzofuryl groups.

The term “substituted aryl or heteroaryl group” means, for example, such a group that is mono- or polysubstituted, especially disubstituted, with a halogen atom, a group —CF₃, —NH₂, —OH or —COOH, an alkyl group containing from 1 to 12 carbon atoms, a group O-alkyl containing from 1 to 8 carbon atoms, a group —(CH₂)ₚR′ or —O—(CH₂)ₚR′ with R′ which represents an aryl group or cycloalkyl and p which is equal to 0 or 1, a nitro function, a group —COOR with R which represents an alkyl group of 1 to 12 carbon atoms or a group —(CH₂)ₚR′ with R′ which represents an aryl group or cycloalkyl and p which is equal to 0, 1, 2 or 3.

The term “amino acid” corresponds to organic compounds having the following structure.

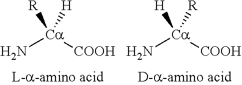

The term “amino acid side chain” thus especially means the group R of the following amino acids: glycine, threonine, serine, aspartic acid, asparagine, cysteine, alanine, proline, valine, isoleucine, histidine, leucine, methionine, phenylalanine, glutamic acid, glutamine, lysine, arginine, tryptophan, tyrosine. The acid, amine or alcohol functions, especially, will preferably be in protected form. Protecting groups for amino acid side chains are well known to those skilled in the art and are especially described in the publication Chem. Rev. 2009, 109, 2455-2504 which is incorporated by reference into the present patent application.

In general, protecting groups for an amino group are well known to those skilled in the art and are described especially in this same publication, or alternatively in Protective Groups in Organic Synthesis, Greene T. W. and Wuts P. G. M., published by John Wiley & Sons, 2006 and in Protecting Groups, Kocienski P. J., 1994, Georg Thieme Verlag. Examples of such groups that may be mentioned include Boc (tert-butyloxycarbonyl), Fmoc (9-fluorenylmethoxycarbonyl) and Z (benzyloxycarbonyl) groups. The presence of such a group at the substituent R1 can increase the hydrophobic nature of the molecule.

The amino acid analogs used in the present invention include, without being limited thereto, homoglutamic acid and homoserine.

The term “azapeptide or azapeptidomimetic” means a peptide or a peptidomimetic in which the CHα is replaced with an N. The term “peptidomimetic” means the replacement of the peptide bond, i.e. the link between two amino acids formed by removal of water between the amine group of one amino acid and the carboxylic acid group of the other amino acid, via an isosteric bond, for example methylamino —CH₂—NH—. The term “cancer” denotes any pathological condition that is typically characterized by deregulated cell growth. Examples of cancers that may be mentioned include carcinomas, lymphomas, blastomas, sarcomas and leukemias, and more specifically epidermoid lung cancer, colon adenocarcinoma, glioma, mesothelioma, breast adenocarcinoma, melanoma, clear-cell kidney cancer, prostate cancer, hepatocarcinoma and multiple myeloma.

The term “treatment” denotes any prophylactic or suppressive therapeutic measure on a disease or disorder leading to a desirable clinical effect or to any beneficial effect, especially including the elimination or decrease of one or more symptoms, or the regression, slowing-down or halting of the progress of the cancer or of the disorder associated therewith.

The term “therapeutically effective amount” denotes any amount of a composition that improves one or more of the characteristic parameters of the cancer.

The synthesis of the compounds of formula (I) is detailed hereinbelow.

The compounds of formula (I) in which X₁═N and Y═C(O) are obtained by a coupling reaction between the compounds of formula (II) and the compounds of formula (III) as presented in SCHEME 1, in which R₂, R₃, X₂, X₃ and X₄ are as defined for (I) and R_{1P} represents either directly the group R₁ in the case where R₂ is an optionally substituted aryl or heteroaryl group, or a hydrogen atom in the case where R₂ represents a side chain optionally in protected form of an amino acid or an amino acid analog.

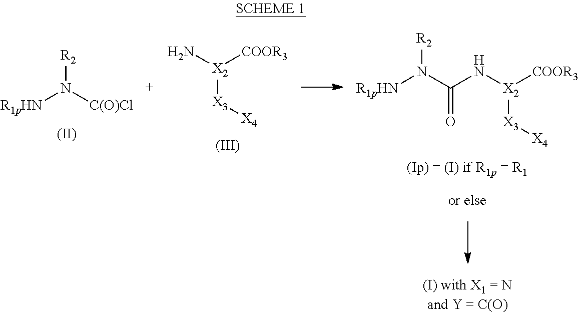

The acid chloride (II) is generated in situ via the action of triphosgene and is coupled, according to a method described in the literature (Journal of Peptide Science, 1997, 3, 429-441), with a compound (III) which corresponds either, in the case where X₂═CH, to a D or L amino acid whose acid function (—COOR₃) and amine function optionally present on the side chain (—X₃-X₄) are protected, or, in the case where X₂═N, to an aza-amino acid whose acid function (—COOR₃) and amine function optionally present on the side chain (—X₃-X₄) are protected.

The compounds of formula (II), in the case where R₂ is an optionally substituted aryl or heteroaryl group, are prepared according to techniques that are well known to those skilled in the art.

SCHEME 2 illustrates their synthesis in the case of the preparation of the compounds of formula (IA), when R₂ is a phenyl substituted with a group —COOR″₅. In SCHEME 2 R₁ and R₅ are as defined for (I) and R₅ₚ represents R₅ or a precursor group of R₅, for example —COOH.

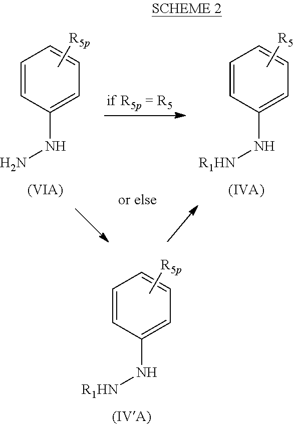

A regioselective acylation of hydrazinobenzoic acid (VIA) is first performed (J. Med. Chem., 1996, 39, 1172-1188). Depending on the nature of the group R₅, and especially in the case where R₅=—COOR″₅ (with R″₅ as defined for the compounds of formula (IA)), an intermediate compound (IV′A) will be formed, in which R₅ₚ=—COOH, which will then be esterified to —COOR″₅, for example via the Wang method (J. Org. Chem. 1977, 42, 1286-1290). The acid chloride (II) is then generated in situ via the action of triphosgene, followed by coupling with compound (III).

The compounds of formula (II), in the case where R₂ represents a side chain, optionally in protected form, of an amino acid or an amino acid analog, are also prepared according to techniques that are well known to those skilled in the art, starting with the compounds of formula (IVB), illustrated in the case where R₂ represents a group —(CH₂)ₘ—COOR₆, with m and R₆ as defined for the compounds of formula (IB):

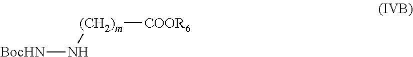

SCHEME 3 illustrates their synthesis in the case of the preparation of the compounds of formula (IB), when R₂ is a —CH₂COOBn.

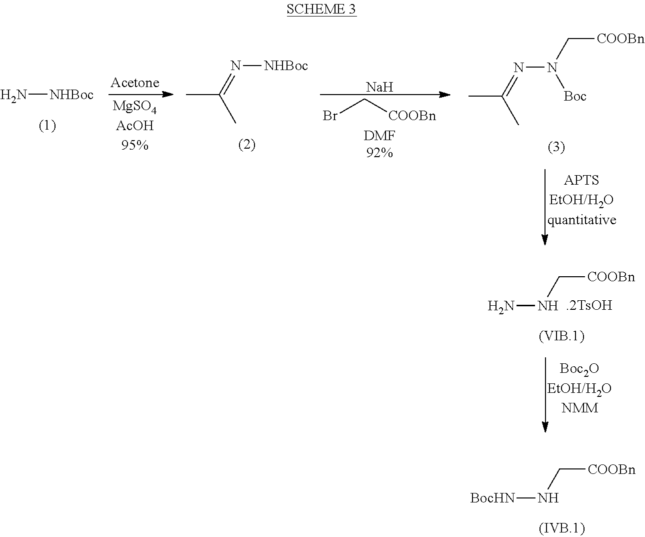

The Nβ Boc hydrazino acetates (IVB.1) are prepared from Boc hydrazine (1). In a first stage, the primary amine of Boc-hydrazine (1) is protected with acetone to form the imine (2). Next, according to a method adapted from that of Meyer (K. Meyer Synlett, 2004, 2355-2356), an alkylation reaction using a bromoacetate leads to the alkyl compound (3). In order simultaneously to deprotect the two amines, para-toluenesulfonic acid is advantageously used to give compound (VIB.1). Protection of the primary amine to give (IVB.1) followed by conversion to the acid chloride and coupling with the compounds (III) are performed according to the same method as in the case of the compounds (IVA).

The compounds of formula (III), may themselves especially be prepared:

either via a Buchwald reaction (Org. Lett., 2001, 3, 3803-3805) between a suitably protected iodoaniline (5) obtained from the iodoaniline (4), according to a method adapted from that of Pati (Synthetic Communications, 2004, 34, 933-40), and Boc-hydrazine, to give the corresponding compound (III). SCHEME 4 below illustrates this preparation method in the case of compound (III.1) where X₂═N, R₃=tert-butyl

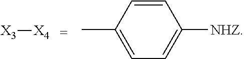

and

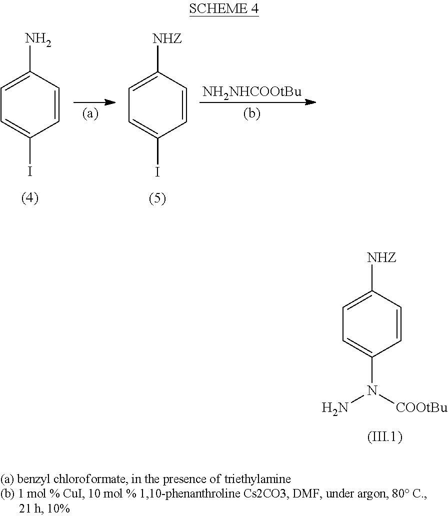

or via a Mitsunobu reaction between the hydrazines β-protected with a phthaloyl group and α-protected with a carbamate group and protected amino alcohols, protected with the preferred groups, on the amine function according to a process described in the literature (Tetrahedron, 1989, 45, 6319-6330 and J. Org. Chem., 2001, 66, 2869-2873). The amino alcohols protected on the amine function are prepared according to the method described in the literature (Tetrahedron 1989, 45, 6319-6330 and Tetrahedron Asymmetry 2003, 14, 139-143). SCHEME 5 below illustrates this preparation method in the case where X₂═N, —COOR₃=Boc and X₃-X₄=—(CH₂)₆NHZ.

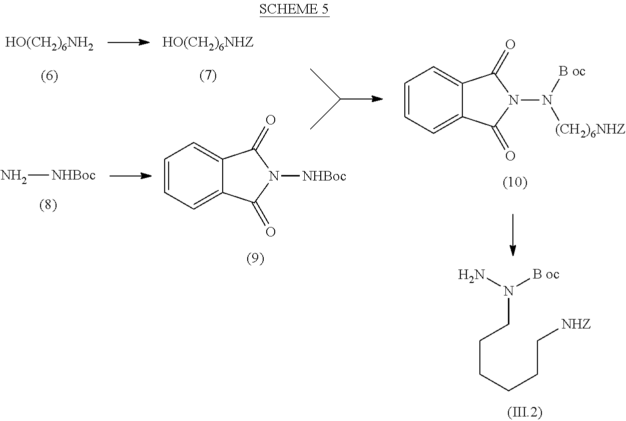

The amine (6) is protected as an amino alcohol (7). In parallel, the Boc-hydrazine (8) is protected with a phthaloyl group to give compound (9). The Mitsunobu reaction between the protected hydrazine (9) and the amino alcohol (7) is followed by deprotection of the aza-amino acid to give compound (III.2).

The compounds of formula (I) in which X₁═CH, X₂═N and Y═CH₂ are prepared via a reductive amination according to the method of Martinez et al. (J. Med. Chem. 1985, 28, 273-278) between a suitably protected amino aldehyde (V) and a compound (III) as described previously, as illustrated in SCHEME 6 hereinbelow.

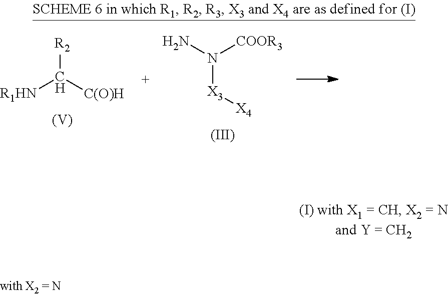

The amino aldehyde (V) may be prepared according to the method of Z. Guo et al. (Bioorg. Med. Chem. 2001, 9, 99-106). SCHEME 7 illustrates the preparation of these compounds in the case where R₁=Boc and R₂=—CH₂—CO₂Bn: compound (V.1) is obtained by reduction of the amino acid (11) to the amino alcohol (12), which is then oxidized.

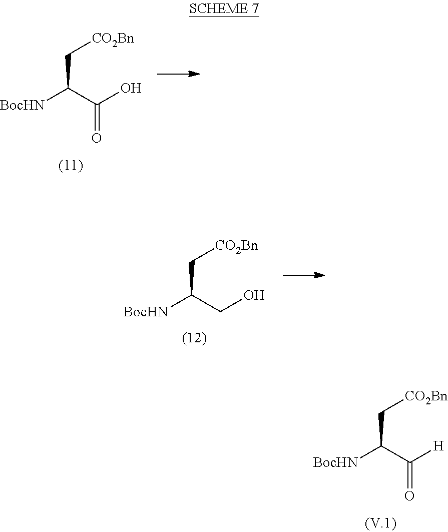

The compounds of formula (I) in which X₁═CH, X2=N and Y═CO are prepared via a peptide coupling reaction according to the method of Bouillon et al. (Tetrahedron 2007, 63, 223-2234) between a suitably protected amino acid (VII) and a compound (III) as described previously, as illustrated in SCHEME 8 below.

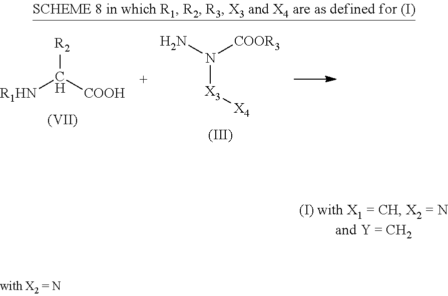

The salts of the compounds according to the invention are prepared according to techniques that are well known to those skilled in the art. The salts of the compounds of formula (I) according to the present invention comprise those with mineral or organic acids or bases that allow suitable separation or crystallization of the compounds of formula (I), and also of the pharmaceutically acceptable salts. Suitable acids that may be mentioned include: oxalic acid or an optically active acid, for example a tartaric acid, a dibenzoyltartaric acid, a mandelic acid or a camphorsulfonic acid, and those that form physiologically acceptable salts, such as the hydrochloride, hydrobromide, sulfate, hydrogen sulfate, dihydrogen phosphate, maleate, fumarate, 2-naphthalenesulfonate, para-toluenesulfonate, mesylate, besylate or isethionate. Suitable bases that may be mentioned include: lysine, arginine, meglumine, benethamine and benzathine, and those that form physiologically acceptable salts, such as the sodium, potassium or calcium salts.

Compounds in hydrated form that may be mentioned, for example, include the semihydrates and monohydrates.

When a compound according to the invention contains one or more asymmetric carbons, the optical isomers of this compound form an integral part of the invention. The present invention comprises the compounds of formula (I) in the form of pure isomers, but also in the form of a mixture of isomers in any proportion. The compounds (I) are isolated in the form of pure isomers via the standard separation techniques: use may be made, for example, of fractional recrystallization of a racemic salt with an optically active acid or base, the principle of which is well known, or standard chromatography techniques on a chiral or achiral phase.

The compounds of formula (I) above also comprise those in which one or more hydrogen, carbon or halogen atoms, especially chlorine or fluorine, have been replaced with the radioactive isotope thereof, for example tritium or carbon-14. Such labeled compounds are useful in research, metabolism or pharmacokinetic studies, or in biochemical tests.

The functional groups that may be present in the molecule of the compounds of formula (I) and in the reaction intermediates may be protected, either in permanent form or in temporary form, with protecting groups that ensure an unequivocal synthesis of the expected compounds. The protection and deprotection reactions are performed according to techniques that are well known to those skilled in the art. The term “temporary or permanent protecting group for amines, alcohols or carboxylic acids” means protecting groups such as those described in Protective Groups in Organic Synthesis, Greene T. W. and Wuts P. G. M., published by John Wiley & Sons, 2006 and in Protecting Groups, Kocienski P. J., 1994, Georg Thieme Verlag.

The compounds of formula (I) described previously show inhibitory activity on one or more of the efflux proteins P-gp and/or BCRP, this activity being demonstrated according to the tests described below. Furthermore, no sign of toxicity is observed with these compounds at the pharmacologically active doses and their toxicity is thus compatible with their use as medicaments.

They may thus be used for potentiating the effect, i.e. increasing the effect, of chemotherapeutic agents, especially anti-infectious or anticancer agents, which have become inactive toward resistant strains via such an efflux mechanism. The term “potentiation” means that by combining a compound of formula (I) and an anti-infectious or anticancer agent, a therapeutic effect is obtained that is higher than that obtained with one or other of the compounds and even higher than the sum of the effects obtained separately.

Thus, the compounds of formulae (I), (IA) and (IB), and also the salts, solvates or hydrates thereof according to the invention, may be used as adjuvants for anticancer or anti-infectious agents or for any other chemotherapeutic treatment in human and/or veterinary medicine, for which the treated body develops resistance of MDR type, so as to restore their activity.

The two treatments, that with the compound of formula (I) and that with the chemotherapy agent, may be simultaneous, sequential, successive or spread over time. The two active principles may be administered, separately, each in a separate pharmaceutical composition, the administration possibly being simultaneous or spread over time, or administered jointly in a single pharmaceutical composition, the administration then being simultaneous.

Various orders of administration or of treatment may be envisioned in the context of an administration spread over time. The compound of formula (I) may be administered before (for example 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly, or after (for example, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the chemotherapy agent.

A subject of the present invention is thus also the compounds of formula (I), and also the pharmaceutically compatible salts thereof, or possible solvates or hydrates, as medicaments, of compositions that may be administered to animals (including man), containing an effective dose of a compound according to the invention or of an acceptable salt, solvate or hydrate thereof, and suitable excipients.

A subject of the present invention is also pharmaceutical compositions containing with suitable excipients, separately or in a single formulation, an effective dose of a compound of formula (I), (IA) or (IB), optionally in the form of a pharmaceutically acceptable salt, solvate or hydrate, and of a chemotherapy agent, preferably chosen from anthracyclines, topoisomerase inhibitors, antimetabolic agents (antifolates), tyrosine kinase inhibitors, antiviral agents (reverse transcriptase inhibitors), antiparasitic agents and antifungal agents.

Said excipients are chosen according to the pharmaceutical form and the desired mode of administration.

In the pharmaceutical compositions of the present invention for oral, sublingual, subcutaneous, intramuscular, intravenous, topical, intratracheal, intranasal, transdermal, rectal or intraocular administration, the active principles of formula (I) above, or the possible salts, solvates and hydrates thereof, may be administered in unit administration forms, as a mixture with pharmaceutically standard supports, to animals and human beings for the prophylaxis or treatment of the above disorders or diseases. The appropriate unit administration forms include oral-route forms such as tablets, gel capsules, powders, granules and oral solutions or suspensions, sublingual, buccal, intratracheal or intranasal administration forms, subcutaneous, intramuscular or intravenous administration forms and rectal administration forms. For topical application, the compounds according to the invention may be used in creams, ointments, lotions or eyewashes.

In order to obtain the effect, the dose of active principle preferably ranges between 1 and 100 mg per kg of body weight and per day. Compound (I) and the anti-infectious or anticancer agent whose effect is to be potentiated are advantageously administered in a ratio of 4 to 1.

When a solid composition is prepared in the form of tablets, the active principle ingredient is mixed with a pharmaceutical vehicle, such as gelatin, starch, lactose, magnesium stearate, talc, gum arabic or the like. The tablets may be coated with sucrose, a cellulose derivative or other suitable materials, or alternatively they may be treated so that they have sustained or delayed activity and so that they continuously release a predetermined amount of active principle.

A preparation as gel capsules is obtained by mixing the active ingredient with a diluent and by pouring the mixture obtained into soft or hard gel capsules.

The pharmaceutical compositions containing a compound of the invention may also be in liquid form, for example solutions, emulsions, suspensions or syrups. The appropriate liquid forms may be, for example, water, organic solvents such as glycerol or glycols, and also mixtures thereof, in varied proportions, in water.

A preparation in the form of a syrup or elixir for administration in the form of drops may contain the active ingredient together with a sweetener, preferably a calorie-free sweetener, methylparaben and propylparaben as antiseptic agent, and also a flavoring and a suitable colorant. The water-dispersible powders or granules may contain the active ingredient as a mixture with dispersants or wetting agents, or suspension agents, for instance polyvinylpyrrolidone, and similarly with sweeteners or flavor enhancers.

Thus, a subject of the present invention is also pharmaceutical compositions containing several active principles in combination, one of which is a compound (I) and the other an anti-infectious or anticancer agent as defined previously.

Moreover, in general, the same preferences as those indicated previously for the compounds (I) and compositions are applicable mutatis mutandis to the medicaments and uses involving these compounds.

The examples below collated in TABLES 2 to 5 illustrate the invention, but have no limiting nature.

The following abbreviations are used:
Et=ethyl; Ac=acetyl; Bn=benzyl; Z=—C(O)OCH₂Ph; Boc=tert-butyloxycarbonyl; tBu=tert-butyl; Ph=phenyl; EtOAc: ethyl acetate; DCM: dichloromethane; DMF: dimethylformamide; DMSO: dimethyl sulfoxide; P.E.: petroleum ether; NMM: N-methylmorpholine; eq.: equivalent(s); IR: infrared; Rf: frontal ratio; DBAD: di-tertbutyl azidodicarboxylate; DEAD: diethyl azidodicarboxylate; DIAD: diisopropyl azidodicarboxylate; Asp: aspartic acid; Glu: glutamic acid; Lys: lysine; EDCI: N-(3-dimethylaminopropyl)-N′-ethylcarbodiimide; HOBt: 1-hydroxybenzotriazole; DCC: dicyclohexylcarbodiimide; DMAP: 4-dimethylaminopyridine.

TABLE 2
(IA)

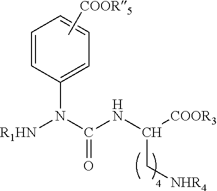

with X₁ = N, Y = —C(O)—, X₂ = —CH—, R₅ = —COOR″₅,
-X₃-X₄- = —(CH₂)₄—NHR₄

| Ex. | R₁ | COOR″₅ | R₃ | R₄ | Stereochemistry |
|---|---|---|---|---|---|
| IA.1 | Boc | o-COOBn | tBu | Z | L |
| IA.2 | Boc | m-COOBn | tBu | Z | L |
| IA.3 | Boc | p-COOBn | tBu | Z | L |
| IA.4 | Boc | m-COOBn | Bn | Z | D |
| IA.5 | Boc | m-COOBn | Bn | Z | L |
| IA.6 | Boc | m-COOC₆H₅ | tBu | Z | L |

TABLE 3

| (IA) | | | | |
|---|---|---|---|---|
| 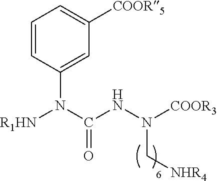 | | | | |
| with X₁ = N, Y = —C(O)—, X₂ = N, R₅ = —COOR″₅, | | | | |
| -X₃-X₄- = (CH₂)₆—NHR₄ | | | | |
| Ex. | R₁ | COOR″₅ | R₃ | R₄ |
| IA.7 | Boc | m-COOBn | Bn | Z |
| IA.8 | Boc | m-COOBn | Bn | Boc |

TABLE 4
(IB)

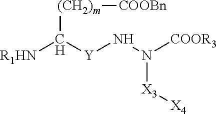

with X₁ = CH, X₂ = N, and R₆ = Bn, stereochemistry L

| Ex. | Y | R₁ | m | R₃ | -X₃-X₄ |
|---|---|---|---|---|---|
| IB.1 | CH₂ | Boc | 1 | tBu | —(CH₂)₆—NHZ |
| IB.2 | CO | Boc | 1 | tBu | —(CH₂)₃—NHZ |
| IB.3 | CO | Boc | 1 | tBu | 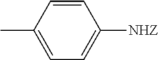 |
| IB.4 | CO | Boc | 2 | tBu | 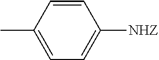 |
| IB.5 | CO | Boc | 1 | tBu | —(CH₂)₅—NHZ |
| IB.6 | CH₂ | Boc | 1 | tBu | 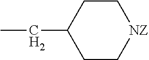 |
| IB.7 | CO | Boc | 1 | tBu | 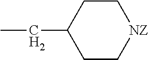 |
| IB.8 | CH₂ | Boc | 1 | tBu | —(CH₂)₅—NHZ |
| IB.9 | CO | Boc | 1 | tBu | —(CH₂)₆—NHZ |
| IB.10 | CH₂ | Boc | 1 | tBu | —(CH₂)₆—NHBoc |
| IB.11 | CH₂ | Boc | 1 | Bn | —(CH₂)₆—NHZ |
| IB.12 | CH₂ | Z | 1 | Bn | —(CH₂)₆—NHZ |
| IB.13 | CH₂ | Boc | 1 | Bn | —(CH₂)₆—NHBoc |

TABLE 5
(IB)

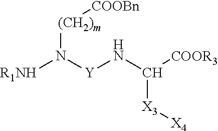

with X₁ = N, X₂ = CH and R₆ = Bn

| Ex. | Y | R₁ | m | R₃ | -X₃₋X₄ | Stereochemistry |
|---|---|---|---|---|---|---|
| IB.14 | CO | Boc | 1 | tBu | —(CH₂)₄—NHZ | L |

### I. EXPERIMENTAL SECTION

### Examples of Synthesis of Compounds

### General Methods

The thin-layer analytical chromatographies are performed on plates of Merck 60 F254 silica on aluminum foil. Detection is performed under UV (254 nm) with revelation by spraying with a solution of ninhydrin in ethanol or of an acidic anisaldehyde solution.
The flash chromatography purifications are performed on Merck 60 Å silica gel (40-63 μm).
The melting points are determined on an Electrothermal 9200 heating-block melting point machine.
The optical rotations are measured using a Bellingham+Stanley Ltd ADP 220 polarimeter or a Jasco P-1010 polarimeter.
The infrared spectra are acquired as a film on a sodium chloride pellet for the oils or after pelletizing in potassium bromide for the solids. The measuring machine is a Perkin-Elmer Spectrum One FT-IR spectrometer.
The proton nuclear magnetic resonance (NMR) spectra are acquired on a Brüker ALS 300 machine (300 MHz) in the solvent specified in parentheses. The carbon spectra are recorded on a Brüker DRX 300 machine (75 MHz). The chemical shifts (•) are expressed in ppm (parts per million). The multiplicities are: singlet (s), doublet (d), triplet (t), quartet (q) and multiplet (m).
The mass spectra were acquired in electrospray mode on a Thermo-Finnegan LCQ-Advantage machine using dichloromethane as solvent.

### EXAMPLE 1

### tert-Butyl ester of (2S)-6-(benzyloxycarbonylamino)-2-[1-(2′-(benzyloxycarbonyl)phenyl)-2-(tert-butoxycarbonyl)hydrazino-carbonylamino]hexanoic acid (IA.1)

### a) 2-[2-(tert-Butoxycarbonyl)hydrazino]benzoic acid (IV′A.1)

To a solution of 3 g of 2-hydrazinobenzoic acid hydrochloride (16 mmol, 1 eq.) in dioxane (1 mL/mmol) is added a solution of 3.81 g of Boc₂O (17.5 mmol, 1.1 eq.) in dioxane (0.4 mL/mmol). The solution is cooled to 0° C. and aqueous 5% Na₂CO₃ solution is added dropwise to pH 8-9. The solution is then warmed to room temperature and stirring is continued for 24 hours. The dioxane is evaporated off. The medium is taken up in water and then acidified with 1N HCl solution to pH 1. The mixture is extracted three times with ethyl acetate. The combined organic phases are dried over sodium sulfate, filtered and evaporated to dryness. The powder obtained is triturated with petroleum ether and then dried under vacuum to give 3.75 g of compound (IV′A.1) (yield 93%).

¹H NMR 300 MHz (DMSO): δ (ppm)=1.41 (s, 9H, C(CH₃)₃); 6.75 (t, J=7.5 Hz, 1H, H₃); 6.86 (d, J=8.1 Hz, 1H, H₅); 7.43 (t, J=7.8 Hz, 1H, H₄); 7.81 (d, J=8.1 Hz, 1H, H₆); 8.88 (s, 1H, NH); 9.05 (s, 1H, NH); 13 (s, 1H, COOH).

IR (KBr): ν_{NH}=3381.6 cm⁻¹; ν_{Csp3-H}=2971.7 cm⁻¹; ν_{C═O}=1697.6 cm⁻¹ and 1676.7 cm⁻¹; ν_{C═C}=1609.1 cm⁻¹ and 1587.2 cm⁻¹.

Melting point=146° C.

### b) Benzyl ester of 2-[2-(tert-butoxycarbonyl)hydrazino]benzoic acid (IVA.1)

1.5 g of N_{β}-Boc-hydrazinobenzoic acid (IV′A.1) (5.94 mmol, 1 eq.) are dissolved in methanol (4.2 mL/mmol) and water (0.42 mL/mmol). The pH is brought to pH 7 by adding aqueous 20% CsCO₃ solution. The solvent is evaporated off and the residue is taken up twice in DMF (1 mL/100 mg of acid) and then re-evaporated. The cesium salt thus isolated is diluted in DMF (2 mL/100 mg of acid), and 0.78 mL of benzyl bromide (6.54 mmol, 1.1 eq.) is added. The reaction mixture is kept stirring for 4 hours at room temperature. The DMF is evaporated off and the mixture obtained is taken up in a large volume of water and then extracted with ethyl acetate. The organic phase is washed with water and then with saturated NaCl solution, dried over sodium sulfate, filtered and evaporated to dryness. The crude product is purified by flash chromatography on silica gel, eluting with a DCM/P.E. mixture (3/1 by volume), to give 1.88 g of a pale yellow oil (yield 96%).

¹H NMR 300 MHz (DMSO): δ (ppm)=1.41 (s, 9H, C(CH₃)₃); 5.34 (s, 2H, CH₂); 6.75-7.89 (m, 9H, Hₐᵣₒₘ); 8.73 (s, 1H, NH); 9.10 (s, 1H, NH).

IR (NaCl): ν_{NH}=3346.2 cm⁻¹; ν_{Csp2-H}=3066.3 cm⁻¹ and 3034.8 cm⁻¹; ν_{Csp3-H}=2978.4 cm⁻¹ and 2934.6 cm⁻¹; ν_{C═O}=1722.9 cm⁻¹ and 1692 cm⁻¹; ν_{C═C}=1606.4 cm⁻¹ and 1585.6 cm⁻¹.

c) To a solution of 250 mg of hydrazinoester (IVA.1) (0.73 mmol, 1 eq.) in distilled DCM (4 mL/mmol) is added 0.35 eq. of triphosgene. The temperature of the reaction medium is reduced to −10° C. and a solution of NMM (1.08 eq.) in distilled DCM (0.5 mL/mmol) is then introduced. The reaction medium is stirred at −10° C. for 1 hour. 270 mg of H-Lys(Z)-OtBu (0.73 mmol, 1 eq.) and a solution of NMM (1.08 eq.) in distilled DCM (0.5 mL/mmol) are then added. The temperature is maintained for 2 hours at −10° C. and then gradually warmed to room temperature. After 20 hours at room temperature, the medium is diluted by adding DCM (30 mL/mmol of hydrazinoester) and then filtered. The organic phase is washed with 0.5M KHSO₄ solution and then with water, dried over sodium sulfate, filtered and evaporated. Purification of the crude product is performed by flash chromatography on silica gel, eluting with an EtOAc/P.E. mixture (1/2 by volume). 250 mg of a yellow oil are obtained (yield 45%).

¹H NMR 300 MHz (CDCl₃): δ (ppm)=0.87 (m, 2H, CH₂—CHα); 1.44 (s, 9H, C(CH₃)₃); 1.48 (s, 9H, C(CH₃)₃); 1.7 (m, 4H, CH₂—(CH₂)₂—CH₂); 3.18 (m, 2H, CH₂—NHZ); 4.46 (m, 1H, CHα); 5.02 (s, broad, 1H, NH); 5.08 (s, 2H, CH₂—C₆H₅); 5.36 (s, 2H, CH₂—C₆H₅); 6.12 (s, broad, 1H, NH); 7.12 (s, broad, 1H, NH); 7.38-8.2 (m, 14H, Hₐᵣₒₘ).

¹³C NMR 75 MHz (CDCl₃): δ (ppm)=11.37; 14.61; 21.45; 22.39; 28.42; 29.6; 30.1; 33.1; 41.19; 53.92; 60.78; 66.83; 67.58; 77.1; 77.53; 77.73; 77.95; 82.27; 127.6; 128.36; 128.44; 128.65; 128.75; 128.85; 129.1; 132.01; 133.98; 135.8; 137.18; 141.64; 155.08; 156.61; 156.86; 166.13; 171.53; 172.13.

IR (NaCl) : ν_{NH}=3349.2 cm⁻¹; ν_{Csp2-H}=3066.1 cm⁻¹ and 3033.8 cm⁻¹; ν_{Csp3-H}=2978.3 cm⁻¹ and 2934.1 cm⁻¹; ν_{C═O}=1725.6 cm⁻¹, 1713.6 cm⁻¹, 1700.9 cm⁻¹ and 1681.7 cm⁻¹; ν_{C═C}=1599.8 cm⁻¹ and 1578.2 cm⁻¹.

[α]_{D}^{25° C.}=+1.68° (16.8 g.L⁻¹, DCM).

HRMS-ESI (m/z): [M+Na] calc. for C₃₈H₄₈N₄O₉, 727.3319; exp., 727.3324.

### EXAMPLE 2

### tert-Butyl ester of (2S)-6-(benzyloxycarbonylamino)-2-[1-(3′-(benzyloxycarbonyl)phenyl)-2-(tert-butoxycarbonyl)hydrazino-carbonylamino]hexanoic acid (IA.2)

### a) 3-[2-(tert-Butoxycarbonyl)hydrazino]benzoic acid (IV′A.2)

According to a procedure identical to that described for compound (IV′A.1), 2.4 g of 3-hydrazinobenzoic acid (16 mmol, 1 eq.) are reacted with 3.8 g of Boc₂O (17 mmol, 1.1 eq.) to give 3.96 g of acid (IV′A.2) (yield 98%).

¹H NMR 300 MHz (DMSO): δ (ppm)=1.42 (s, 9H, C(CH₃)₃); 6.88 (m, 1H, H₄); 7.27 (m, 3H, H₂—H₅—H₆); 7.83 (s, 1H, NH); 8.86 (s, 1H, NH); 12.78 (s, 1H, COOH).

IR (KBr): ν_{NH}=3315.6 cm⁻¹; ν_{Csp3-H}=2982.0 cm⁻¹; ν_{C═O}=1706.4 cm⁻¹ and 1689.2 cm⁻¹; ν_{C═C}=1611.2 cm⁻¹ and 1594.4 cm⁻¹.

Melting point=150° C.

### b) Benzyl ester of 3-[2-(tert-butoxycarbonyl)hydrazino]benzoic acid (IVA.2)

According to a procedure identical to that described for compound (IVA.1), 406 mg of compound (IV′A.2) (1.61 mmol, 1 eq.) and 0.211 mL of benzyl bromide (1.77 mmol, 1.1 eq.) lead, after purification by flash chromatography on silica gel, eluting with an EtOAc/P.E. mixture (1:5) by volume, to 283 mg of a yellow oil (yield 51%).

¹H NMR 300 MHz (DMSO): δ (ppm)=1.40 (s, 9H, C(CH₃)₃); 5.32 (s, 2H, CH₂); 7.25-7.50 (m, 9H, Hₐᵣₒₘ); 7.90 (s, 1H, NH); 8.88 (s, 1H, NH).

IR (NaCl): ν_{NH}=3334.5 cm⁻¹; ν_{Csp2-H}=3066.1 cm⁻¹ and 3034.5 cm⁻¹; ν_{Csp3-H}=2979.2 cm⁻¹ and 2934.7 cm⁻¹; ν_{C═O}=1714.9 cm⁻¹ and 1699.1 cm⁻¹; ν_{C═C}=1608.2 cm⁻¹ and 1595.1 cm⁻¹.

c) According to a procedure identical to that described for compound (IA.1), 270 mg of hydrazinoester (IVA.2) (0.79 mmol, 1 eq.) and 295 mg of H-Lys(Z)-OtBu (0.79 mmol, 1 eq.) lead, after purification by flash chromatography on silica gel, eluting with an EtOAc/P.E. mixture (1/3) and then (1/2) by volume, to 220 mg of a yellow oil (yield 40%).

¹H NMR 300 MHz (CDCl₃): δ (ppm)=1.18 (m, 2H, CH₂—CHα); 1.35 (s, 9H, C(CH₃)₃); 1.38 (s, 9H, C(CH₃)₃); 1.40-1.62 (m, 4H, CH₂—(CH₂)₂—CH₂); 3.10 (m, 2H, CH₂—NHZ); 4.35 (m, 1H, CHα); 5.02 (5, 2H, CH₂—C₆H₅); 5.28 (s, 2H, CH₂—C₆H₅); 5.85 (s, broad, 1H, NH); 6.95 (s, broad, 1H, NH); 7.2-8.0 (m, 14H, Hₐᵣₒₘ).

¹³C NMR 75 MHz (CDCl₃): δ (ppm)=22.14; 28.05; 28.09; 29.34; 32.49; 40.75; 53.71; 66.58; 66.92; 82.18; 82.56; 127.25; 128.07; 128.30; 128.52; 128.63; 129.00; 129.14; 130.99; 135.92; 136.72; 142.06; 154.75; 155.29; 156.57; 165.88; 171.93.

IR (NaCl): ν_{NH}=3334 cm⁻¹; ν_{Csp2-H}=3066.1 cm⁻¹ and 3034.3 cm⁻¹; ν_{Csp3-H}=2978.3 cm⁻¹ and 2927.4 cm⁻¹; ν_{C═O}=1726.6 cm⁻¹, 1714.9 cm⁻¹, 1695.5 cm⁻¹, and 1679.1 cm⁻¹; ν_{C═C}=1604.5 cm⁻¹ and 1587.8 cm⁻¹.

[α]_{D}^{25.7°C.}=+4° (10 g.L⁻¹, DCM).

HRMS-ESI (m/z): [M+Na] calc. for C₃₈H₄₈N₄O₉, 727.3319; exp., 727.3324.

### EXAMPLE 3

### tert-Butyl ester of (2S)-6-(benzyloxycarbonylamino)-2-[1-(4′-(benzyloxycarbonyl)phenyl)-2-(tert-butoxycarbonyl)hydrazino-carbonylamino]hexanoic acid (IA.3)

### a) 4-[2-(tert-Butoxycarbonyl)hydrazino]benzoic acid (IVA′.3)

According to a procedure identical to that described for compound (IV′A.1), 5 g of 4-hydrazinobenzoic acid (26 mmol, 1 eq.) are reacted with 6.54 g of Boc₂O (30 mmol, 1.1 eq.) to give 6.7 g of acid (IVA′.3) (yield 100%).

¹H NMR 300 MHz (DMSO): δ (ppm)=1.42 (s, 9H, C(CH₃)₃); 6.65 (d, J=8.7 Hz, 2H, H₃, H₅); 7.74 (d, J=8.4 Hz, 2H, H₂, H₆); 8.24 (s, 1H, NH); 8.92 (s, 1H, NH); 12.23 (s, 1H, COOH).

IR (KBr): ν_{NH}=3401.4 cm⁻¹; ν_{Csp3-H}=2981.5 cm⁻¹; ν_{C═O}=1700.9 cm⁻¹ and 1683.2 cm⁻¹; ν_{C═C}=1606.2 cm⁻¹.

Melting point=144° C.

### b) Benzyl ester of 4-[2-(tert-butoxycarbonyl)hydrazino]benzoic acid (IVA.3)

According to a procedure identical to that described for compound (IVA.1), 1 g of compound (IVA′.3) (3.96 mmol, 1 eq.) and 0.51 mL of benzyl bromide (4.36 mmol, 1.1 eq.) lead, after purification by flash chromatography on silica gel, eluting with an EtOAc/P.E. mixture (1/2 by volume), to 800 mg of a yellow oil (yield 58%).

NMR 300 MHz (DMSO): δ (ppm)=1.42 (s, 9H, C(CH₃)₃); 5.28 (s, 2H, CH₂); 6.67-7.84 (m, 9H, Hₐᵣₒₘ); 8.35 (s, 1H, NH); 9 (s, 1H, NH).

IR (NaCl): ν_{NH}=3313.9 cm⁻¹; ν_{Csp2-H}=3067 cm⁻¹ and 3034.5 cm⁻¹; ν_{Csp3-H}=2983 cm⁻¹ and 2934.6 cm⁻¹; ν_{C═O}=1709.1 cm⁻¹ and 1688.9 cm⁻¹; ν_{C═C}=1609.4 cm⁻¹.

c) According to a procedure identical to that described for compound (IA.1), 244 mg of hydrazinoester (IVA.3) (0.71 mmol, 1 eq.) and 260 mg of H-Lys(Z)-OtBu (0.71 mmol, 1 eq.) lead, after purification by flash chromatography on silica gel, eluting with an EtOAc/P.E. mixture (1/2 by volume), to 150 mg of a beige-colored powder (yield 30%).

¹H NMR 300 MHz (CDCl₃): δ (ppm)=0.89 (m, 2H, CH₂—CHα); 1.48 (s, 18H, C(CH₃)₃, C(CH₃)₃); 1.68 (m, 4H, CH₂—(CH₂)₂—CH₂); 3.18 (m, 2H, CH₂—NHZ); 4.46 (m, 1H, CHα); 5.0 (s, broad, 1H, NH); 5.09 (s, 2H, CH₂—C₆H₅); 5.36 (s, 2H, CH₂—C₆H₅); 6.1 (s, broad, 1H, NH); 7.08 (s, broad, 1H, NH); 7.8-8.1 (m, 14H, Hₐᵣₒₘ).

¹³C NMR 75 MHz (CDCl₃): δ (ppm)=14.60; 21.46; 22.44; 28.21; 28.40; 28.45; 29.69; 30.09; 31.84; 32.69; 36.89; 41.06; 54.05; 60.81; 66.92; 66.99; 77.10; 77.53; 77.73; 77.95; 82.58; 82.92; 121.85; 126.53; 128.43; 128.53; 128.60; 128.88; 128.98; 130.86; 136.49; 137.03; 146.45; 154.97; 155.36; 156.96; 163.05; 166.3; 172.28.

IR (KBr): ν_{NH}=3389.9 cm⁻¹; ν_{Csp2-H}=3068.2 cm⁻¹ and 3035.4 cm⁻¹; ν_{Csp3-H}=2978.1 cm⁻¹ and 2932.6 cm⁻¹; ν_{C═O}=1719.3 cm⁻¹, 1705.5 cm⁻¹, 1672.2 cm⁻¹ and 1665.2 cm⁻¹; ν_{C═C}=1607.4 cm⁻¹.

[α]_{D}^{25° C.}=+2.15° (13.9 g.L⁻¹, DCM).

Melting point=106° C.

HRMS-ESI (m/z): [M+Na] calc. for C₃₈H₄₈N₄O₉, 727.3319; exp., 727.3315.

### EXAMPLE 4

### Benzyl ester of (2R)-6-(benzyloxycarbonylamino)-2-[1-(3′-(benzyloxycarbonyl)phenyl)-2-(tert-butoxycarbonyl)hydrazino-carbonylamino]hexanoic acid (IA.4)

To a solution of 500 mg of hydrazinoester (IVA.2) (1.46 mmol, 1 eq.) in distilled DCM (4 mL/mmol) is added 0.35 eq. of triphosgene. The temperature of the reaction medium is reduced to −10° C. and then a solution of NMM (2 eq.) in distilled DCM (0.5 mL/mmol) is introduced. The reaction medium is stirred at −10° C. for 1 hour. 600 mg of HCl.H-D-Lys(Z)-OBn (1.46 mmol, 1 eq.) and a solution of NMM (2 eq.) in distilled DCM (0.5 mL/mmol) are then added. The temperature is maintained for 2 hours at −10° C. and then gradually warmed to room temperature. After 20 hours at room temperature, the medium is diluted by adding DCM (30 mL/mmol of hydrazinoester) and then filtered. The organic phase is washed with 0.5M KHSO₄ solution and then with water, dried over sodium sulfate, filtered and evaporated. Purification of the crude product is performed by flash chromatography on silica gel, eluting with an EtOAc/cyclohexane mixture (3/7 by volume). 362 mg of a yellow paste are obtained (yield 34%).

¹H NMR 300 MHz (DMSO): δ (ppm)=0.88-1.20 (m, 2H, CH₂—CHα); 1.40 (s, 9H, COO(CH₃)₃); 1.60-1.93 (m, 4H, CH₂—(CH₂)₂—CHα); 3.07-3.14 (m, 2H, CH₂—NHZ); 4.55-4.63 (m, 1H, CHα); 5.06 (s, 2H, CH₂-Ph); 5.17 (s, 2H, CH₂-Ph); 5.33 (s, 2H, CH₂-Ph); 5.35 (s, 2H, CH₂Ph); 7.12 (s broad, 1H, NH); 7.30-7.43 (m, 20H, Hₐᵣₒₘ); 7.70 (s broad, 1H, NH).

¹³C NMR 75 MHz (DMSO): δ (ppm)=28.73; 29.80; 30.48; 31.11; 40.94; 54.24; 65.97; 66.66; 67.14; 81.28; 125.55; 127.48; 128.57; 128.81; 128.88; 128.93; 129.09; 129.18; 129.26; 129.36; 129.38; 129.54; 130.56; 136.91; 138.13; 143.79; 150.01; 155.32; 156.44; 156.91; 166.25; 173.29.

IR (KBr): ν_{NH}=3344 cm⁻¹; ν_{Csp2 Harom}=3065 cm⁻¹ and 3033 cm⁻¹; ν_{C═O}=1715 cm⁻¹; ν_{C═O ester}=1248 cm⁻¹ and 1158 cm⁻¹.

[α]_{D}^{25° C.}=+3° (5 g.L⁻¹, DCM).

HRMS-ESI (m/z): [M+Na] calc. for C₄₁H₄₆N₄O₉, 761.3162; exp., 761.3163.

### EXAMPLE 5

### Benzyl ester of (2S)-6-(benzyloxycarbonylamino)-2-[1-(3′-(benzyloxycarbonyl)phenyl)-2-(tert-butoxycarbonyl)hydrazino-carbonylamino]hexanoic acid (IA.5)

According to a procedure identical to that described for compound (IA.4), 500 mg of hydrazinoester (IVA.2) (1.46 mmol, 1 eq.) and 610 mg of HCl.H-Lys(Z)-OBn (1.46 mmol, 1 eq.) lead, after purification by flash chromatography on silica gel, eluting with an EtOAc/cyclohexane mixture (3/7 by volume), to 320 mg of a yellow paste (yield 30%).

¹H NMR 300 MHz (DMSO): δ (ppm)=1.31 (m, 2H, CH₂—CHα); 1.38 (s, 9H, COO(CH₃)₃); 1.61-1.77 (m, 4H, CH₂—(CH₂)₂—CHα); 2.94-2.96 (m, 2H, CH₂—NHZ); 4.19-4.21 (m, 1H, CHα); 4.99 (s, 2H, CH₂-Ph); 5.13 (s, 2H, CH₂-Ph); 5.35 (s, 2H, CH₂Ph); 7.21-7.72 (m, 19H, Hₐᵣₒₘ).

¹³C NMR 75 MHz (DMSO): δ (ppm)=27.21; 28.13; 28.73; 29.80; 30.48; 31.10; 40.93; 54.23; 65.97; 66.66; 67.14; 81.27; 128.57; 128.81; 128.88; 128.93; 129.00; 129.18; 129.23; 129.26; 129.38; 130.27; 130.56; 136.79; 136.91; 138.13; 143.79; 155.32; 156.45; 156.91; 166.25; 173.04.

IR (KBr): ν_{NH}=3354 cm⁻¹; ν_{Csp2 Harom}=3066 cm⁻¹ and 3034 cm⁻¹; ν_{C═O}=1723 cm⁻¹; ν_{C—O ester}=1248 cm⁻¹ and 1156 cm⁻¹

[α]_{D}^{25° C.}=−3° (5 g.L⁻¹, DCM).

HRMS-ESI (m/z): [M+Na] calc. for C₄₁H₄₆N₄O₉, 761.3162; exp., 761.3160.

### EXAMPLE 6

### tert-Butyl ester of (2S)-6-(benzyloxycarbonylamino)-2-[1-(3′-(phenyloxycarbonyl)phenyl)-2-(tert-butoxycarbonyl)hydrazino-carbonylamino]hexanoic acid (IA.6)

### a) Phenyl ester of 3-(2-tert-butoxycarbonylhydrazino)benzoic acid (IVA.6)

To a solution of 780 mg (3 mmol, 1 eq.) of compound (IV′A.2) in DCM (3 mL/mmol) and DMF (minimum amount for dissolution), are added 618 mg (3 mmol, 1 eq.) of DCC and 873 mg (9 mmol, 3 eq.) of phenol. After stirring for 15 minutes at room temperature, 18 mg (0.15 mmol, 0.05 eq.) of DMAP are introduced. The reaction mixture is stirred at room temperature for 4 hours and then filtered. The filtrate is evaporated to dryness and then taken up in 30 mL of EtOAc. The organic phase is washed twice with 0.1N HCl solution, with 0.1N NaHCO₃ solution and with saturated NaCl solution, dried over sodium sulfate, filtered and evaporated. Purification of the crude product is performed by flash chromatography on silica gel, eluting with an EtOAc/cyclohexane mixture (1/9 by volume).

¹H NMR 300 MHz (DMSO): δ (ppm)=1.41 (s, 9H, C(CH₃)₃); 6.98-7.50 (m, 9H, Hₐᵣₒₘ); 7.97 (s, 1H, NH); 8.93 (s, 1H, NH).

b) According to a procedure identical to that described for compound (IA.1), 300 mg of hydrazinoester (IVA.6) (0.9 mmol, 1 eq.) and 340 mg of H-Lys(Z)-OtBu (0.9 mmol, 1 eq.) lead, after purification by flash chromatography on silica gel, eluting with an EtOAc/cyclohexane mixture (3/7 by volume), to 130 mg of a yellow solid.

¹H NMR 300 MHz (CDCl₃): δ (ppm)=1.19 (m, 2H, CH₂—CHα); 1.39 (s, 9H, C(CH₃)₃); 1.41 (s, 9H, C(CH₃)₃); 1.50-1.72 (m, 4H, CH₂—(CH₂)₂—CH₂); 2.97 (m, 2H, CH₂—NHZ); 4.04 (m, 1H, CHα); 4.99 (s, 2H, CH₂—C₆H₅); 7.23-7.94 (m, 14H, Hₐᵣₒₘ); 8.14 (s, broad, 1H, NH).

IR (KBr): ν_{NH}=3378.6 cm⁻¹; ν_{Csp2 Harom}=3066 cm⁻¹ and 3034 cm⁻¹; ν_{Csp3-H}=2978.2 cm⁻¹ and 2931.7 cm⁻¹; ν_{C═O}=1737.6 cm⁻¹ and 1700.9 cm⁻¹; ν_{C—O ester}=1246.2 cm⁻¹ and 1158.4 cm⁻¹.

[α]_{D}^{25° C.}=0.3° (5 g.L⁻¹, DCM).

HRMS-ESI (m/z): [M+Na] calc. for C₃₇H₄₆N₄O₉, 713.3162; exp., 713.3162.

### EXAMPLE 7

### Benzyl ester of 2-[1-(3′-(benzyloxycarbonyl)phenyl)-2-(tert-butoxycarbonyl)hydrazinocarbonyl]-1-(6-benzyloxycarbonyl-aminohexyl)hydrazinocarboxylic acid (IA.7)

### a) 1-Benzyloxycarbonyl-1-(6-benzyloxycarbonylaminohexyl)-hydrazine (III.3)

Compound (III.3) is obtained according to a procedure identical to that followed for the synthesis of (III.2) described hereinbelow, using benzyloxycarbonylhydrazine and 6-(benzyloxycarbonylamino)hexanol as starting materials.

¹H NMR 300 MHz (DMSO): δ (ppm)=1.39-1.34 (m, 6H, (CH₂)₃); 1.51-1.46 (m, 2H, CH₂); 2.98-2.92 (q, 2H, CH₂—NHZ); 3.31-3.26 (t, 2H, CH₂—N—NH₂); 4.57 (s, 2H, NH₂); 5.00 (s, 2H, CH₂-Ph); 5.06 (s, 2H, CH₂-Ph); 7.37-7.20 (m, 10H, Hₐᵣₒₘ).

IR (KBr): ν_{NH}=3306 cm⁻¹; ν_{Csp2 Harom}=3061 cm⁻¹ and 3030 cm⁻¹; ν_{C═O}=1676 cm⁻¹; ν_{C═C}=1535 cm⁻¹; ν_{C—O ester}=1257 cm⁻¹ and 1190 cm⁻¹.

b) According to a procedure identical to that described for compound (IA.4), 850 mg of hydrazinoester (IVA.2) (2.5 mmol, 1 eq.) and 990 mg of compound (III.3) (2.5 mmol, 1 eq.) lead, after purification by flash chromatography on silica gel, eluting with an EtOAc/cyclohexane mixture (3/7 by volume), to 244 mg of an oil.

¹H NMR 300 MHz (DMSO): δ (ppm)=1.19-1.30 (m, 6H, (CH₂)₃); 1.38 (s, 9H, COO(CH₃)₃); 1.49 (m, 2H, CH₂); 2.98-2.92 (q, 2H, CH₂—NHZ); 3.41 (t, 2H, CH₂—N—NH₂); 4.99 (s, 2H, CH₂-Ph); 5.10 (s, 2H, CH₂-Ph); 5.35 (s, 2H, CH₂Ph); 7.33-7.50 (m, 19H, Hₐᵣₒₘ).

IR (NaCl): ν_{NH}=3308.0 cm⁻¹; ν_{Csp2 Harom}=3065.9 cm⁻¹ and 3034.0 cm⁻¹; ν_{Csp3-H}=2978.2 cm⁻¹ and 2932.8 cm⁻¹; ν_{C═O}=1715.3 cm⁻¹; ν_{C—O ester}=1251.3 cm⁻¹ and 1161.1 cm⁻¹.

HRMS-ESI (m/z): [M+Na] calc. for C₄₂H₄₉N₅O₉, 790.3428; exp., 790.3429.

### EXAMPLE 8

### Benzyl ester of 2-[1-(3′-(benzyloxycarbonyl)phenyl)-2-(tert-butoxycarbonyl)hydrazinocarbonyl]-1-(6-tert-butyloxycarbonyl-aminohexyl)hydrazinocarboxylic acid (IA.8)

### a) 1-Benzyloxycarbonyl-1-(6-tert-butoxycarbonylaminohexyl)-hydrazine (III.4)

Compound (III.4) is obtained according to a procedure identical to that followed for the synthesis of (III.2) described hereinbelow, using benzyloxycarbonylhydrazine and 6-(tert-butoxycarbonylamino)hexanol as starting materials and replacing the DIAD with DBAD in the Mitsunobu reaction.

¹H NMR 300 MHz (DMSO): δ (ppm)=1.35 (s, 9H, COO(CH₃)₃); 1.20-1.50 (m, 8H, CH₂—(CH₂)₄—CH₂); 2.87 (q, 2H, CH₂—NHZ); 3.28 (t, 2H, CH₂—N—NH₂); 4.57 (s, 2H, NH₂); 5.06 (s, 2H, CH₂-Ph); 6.75 (s, 1H, NH); 7.33 (m, 5H, Hₐᵣₒₘ).

IR (KBr): ν_{NH}=3345 cm⁻¹; ν_{Csp2 Harom}=3065 cm⁻¹ and 3033 cm⁻¹; V_{Csp3}=2975 cm⁻¹ and 2933 cm⁻¹; ν_{C═O}=1696 cm⁻¹; ν_{C═C}=1523 cm⁻¹; ν_{C—O ester}=1250 cm⁻¹ and 1174 cm⁻¹.

b) According to a procedure identical to that described for compound (IA.4), 850 mg of hydrazinoester (IVA.2) (2.5 mmol, 1 eq.) and 913 mg of compound (III.4) (2.5 mmol, 1 eq.) lead, after purification by flash chromatography on silica gel, eluting with an EtOAc/cyclohexane mixture (3/7 by volume), to 50 mg of a brown paste.

¹H NMR 300 MHz (DMSO): δ (ppm)=1.23-1.30 (m, 6H, (CH₂)₃); 1.36 (s, 9H, COO(CH₃)₃); 1.39 (s, 9H, COO(CH₃)₃); 1.48 (m, 2H, CH₂); 2.86 (q, 2H, CH₂—NHZ); 3.41 (t, 2H, CH₂—N—NH₂); 5.09 (s, 2H, CH₂-Ph); 5.35 (s, 2H, CH₂-Ph); 7.28-7.47 (m, 14H, Hₐᵣₒₘ).

IR (NaCl): ν_{NH}=3325.0 cm⁻¹; ν_{Csp2 Harom}=3065.9 cm⁻¹ and 3027.4 cm⁻¹; ν_{Csp3-H}=2920.8 cm⁻¹; ν_{C═O}=1704.9 cm⁻¹ and 1684.5 cm⁻¹.

HRMS-ESI (m/z): [M+Na] calc. for C₃₉H₅₁N₅O₉, 756.3584; exp., 756.3585.

### EXAMPLE 9

### Benzyl ester of (3S)-4-[2-(6-benzyloxycarbonylaminohexyl)-2-tert-butoxycarbonylhydrazino]-3-tert-butoxycarbonylaminobutyric acid (IB.1)

### a) 6-Benzyloxycarbonylaminohexan-1-ol (7)

1 g of 6-aminohexanol (6) (8.53 mmol, 1 eq.) and 466 mg Na₂CO₃ (4.4 mmol, 2.2 eq.) are dissolved in 8 mL of water and 4 mL of THF. The medium is cooled to 5° C. 1.33 mL of benzyl chloroformate (9.4 mmol, 1.1 eq.), dissolved in 2.5 mL of THF, are then added dropwise. The reaction is left stirring for 2 hours 45 minutes at room temperature. The THF is evaporated off. The solid formed is filtered off under vacuum, washed with water, and then dried under vacuum to give 1.848 g of a white solid (yield 86%)

¹H NMR 300 MHz (CDCl₃): δ (ppm)=1.20-1.50 (m, 8H, CH₂—(CH₂)₄—CH₂); 3.14 (m, 2H, CH₂—NH); 3.55 (m, 2H, CH₂—OH); 4.70 (s, broad, 1H, NH); 5.05 (s, 2H, CH₂Ph); 7.30 (m, 5H, aromatic).

IR (KBr): ν_{CH}=3384 cm⁻¹; ν_{NH}=3335 cm⁻¹; ν_{Csp2 Harom}=3061 cm⁻¹ and 3030 ν_{Csp3}=2942 cm⁻¹ and 2859 cm⁻¹; ν_{C═O}=1685 cm⁻¹; ν_{C═C}=1530 cm⁻¹ and 1464 cm⁻¹; ν_{C—O ester}=1290 cm⁻¹ and 1253 cm⁻¹.

Melting point=86° C.

### b) N-(tert-Butoxycarbonylamino)phthalimide (9)

Phthalic anhydride (3.36 g, 22.7 mmol, 1 eq.) and tert-butyl carbazate (3 g, 22.7 mmol, 1 eq.) are placed in 65 mL of toluene in a one-necked round-bottomed flask equipped with Dean-Stark apparatus. The solution is heated to reflux. After 2 hours 30 minutes, the reaction medium is cooled to room temperature, and then to 0° C. The product precipitates and is filtered off under vacuum. It is then recrystallized from ethyl acetate to give 5.6 g of a translucent white crystalline solid (yield 94%).

¹H NMR 300 MHz (CDCl₃): δ (ppm)=1.52 (s, 9H, COOC(CH₃)₃); 6.57 (s, 1H, NH); 7.87 (m, 4H, aromatic).

IR (KBr): ν_{NH}=3323 cm⁻¹; ν_{Csp2 Harom}=3092 cm ⁻¹; ν_{Csp3}=2984 cm⁻¹; ν_{C═O}=1798 cm⁻¹ and 1736 cm⁻¹; ν_{C═C}=1530 cm⁻¹.

Melting point=188-190° C. (lit.: 184-186° C.)

### c) N-[(6-Benzyloxycarbonylaminohexyl)(tert-butoxycarbonyl)-amino]phthalimide (10)

N-(tert-Butoxycarbonylamino)phthalimide (9) (1.86 g, 7.10 mmol, 1 eq.), the protected amino alcohol (7) (1.78 g, 7.10 mmol, 1 eq.) and triphenylphosphine (1.5 eq.) are dissolved in anhydrous THF (10 mL/mmol of phthalimide) at 0° C., under argon. 1.5 eq. of DIAD are added. Stirring is maintained at 0° C. for 30 minutes, and then at room temperature. The THF is then evaporated off. The residue is purified by flash chromatography on silica gel (EtOAc/P.E.: 1/1 by volume) to give 2.71 g of an orange oil (yield 77%).

¹H NMR 300 MHz (CDCl₃): δ (ppm)=1.10-1.30 (m, 8H, CH₂—(CH₂)₄—CH₂); 1.40 (s, 9H, COOC(CH₃)₃); 3.10 (m, 2H, CH₂—NH); 3.55 (m, 2H, CH₂—N—N); 4.05 (m, 1H, NH); 5.05 (s, 2H, CH₂-Ph); 6.30 (s, broad, 1H, NH); 7.30 (m, 5H, phenyl); 7.75 (m, 4H, aromatic phthalyl),

IR (NaCl): ν_{NH}=3347 cm⁻¹; ν_{Csp2 Harom}=3065 cm⁻¹ and 3032 cm⁻¹; ν_{Csp3}=2980 cm⁻¹ and 2936 cm⁻¹; ν_{C═O}=1796 cm⁻¹ and 1737 cm⁻¹; ν_{C═C}=1530 cm⁻¹; ν_{C—O ester}=1246 cm⁻¹ and 1157 cm⁻¹.

### d) 1-tert-butyloxycarbonyl-1-(6-benzyloxycarbonylaminohexyl)-hydrazine (III.2)

The phthaloyl derivative (10) (1.5 g, 3.03 mmol, 1 eq.) is dissolved in EtOH (1 mL per 60 mg). The medium is cooled to −20° C. Hydrazine monohydrate (1.5 eq.) is then added. The medium is left stirring at −20° C. for 30 minutes, and then at room temperature for 3 hours. The white solid formed is filtered off. The filtrate is evaporated and then purified by flash chromatography on silica gel (EtOAc/P.E.: 4/6 by volume) to give 734 mg of a yellow oil (yield 67%).

¹H NMR 300 MHz (CDCl₃): δ (ppm)=1.15-1.50 (m, 18H, CH₂—(CH₂)₄—CH₂ and COOC(CH₃)₃); 3.10 (q, J=6.8 Hz and J=6.4 Hz, 2H, CH₂—NHZ); 3.28 (t, J=6.8 Hz, 2H, CH₂—N—NH₂); 3.85 (s, broad, 2H, NH₂); 4.72 (s, broad, 1H, NHZ); 5.03 (s, 2H, CH₂Ph); 7.30 (m, 5H, aromatic).

IR (NaCl): ν_{NH}=3335 cm⁻¹; ν_{Csp2 Harom}=3065 cm⁻¹ and 3033 cm⁻¹; ν_{Csp3}=2975 cm⁻¹ and 2933 cm⁻¹; ν_{C═O}=1694 cm⁻¹; ν_{C═C}=1532 cm⁻¹ and 1455 cm⁻¹; ν_{C—O ester}=1255 cm⁻¹, 1167 cm⁻¹ and 1141 cm⁻¹.

### e) Benzyl ester of 3-tert-butoxycarbonylamino-4-hydroxybutanoic acid (12)

2 g of Boc-Asp(OBn)-OH (11) (6.2 mmol, 1 eq.) are dissolved in 100 mL of anhydrous THF. The medium is placed under argon and cooled to −15° C. 3.5 mL of triethylamine (24.8 mmol, 4 eq.) and 854 μL of isobutyl chloroformate (6.2 mmol, 1 eq.) are then added. After stirring for 20 minutes, the reaction medium is poured dropwise into a solution of NaBH₄ (400 mg, 10.54 mmol, 1.7 eq.) dissolved in 40 mL of THF and 10 mL of MeOH under argon at −78° C. The medium is left stirring for 2 hours. 150 mL of 1M HCl solution are then added. The aqueous phase is extracted three times with EtOAc. The organic phase is washed three times with saturated Na₂CO₃ solution, dried over anhydrous sodium sulfate, evaporated and then purified by flash chromatography on silica gel (EtOAc/P.E.: elution gradient 1/3 to 1/1 by volume) to give 1.043 g of an orange/brown oil (yield 56%).

¹H NMR 300 MHz (CDCl₃): δ (ppm)=1.38 (s, 9H, COOC(CH₃)₃); 2.44 (s, broad, 1H, OH); 2.62 (d, J=6.4 Hz, 2H, CH₂COO); 3.62 (s, 2H, CH₂OH); 3.95 (m, 1H, CHα); 5.05 (s, 2H, CH₂Ph); 5.15 (s, broad, 1H, NH); 7.28 (m, 5H, aromatic).

IR (NaCl): ν_{NH and OH}=3380 cm⁻¹; ν_{Csp2 Harom}=3066 cm⁻¹ and 3034 cm⁻¹; ν_{Csp3}=2977 cm⁻¹ and 2933 cm⁻¹, ν_{C═O}=1713 cm⁻¹; ν_{C═C}1515 cm⁻¹ and 1456 cm⁻¹; ν_{C—O ester}=1249 cm⁻¹ and 1166 cm⁻¹.

### f) Benzyl ester of 3-tert-butoxycarbonylamino-4-oxobutanoic acid (V.1)

To a solution of 1.03 mL of oxalyl chloride (11.75 mmol, 2.5 eq.) in 9 mL of freshly distilled DCM, under argon at −78° C., are added dropwise 1.67 mL of DMSO (23.5 mmol, 5 eq.). The medium is left stirring for 15 minutes under these conditions. 1.45 g of compound (12) (4.70 mmol, 1 eq.), dissolved in 20 mL of freshly distilled DCM, are added. Stirring is maintained for 1 hour under the same conditions. 5.9 mL of freshly distilled triethylamine (42.3 mmol, 9 eq.) are then added dropwise. Stirring is continued for 30 minutes at −78° C. and then for 15 minutes at room temperature. The medium is diluted by adding DCM and H₂O. The aqueous phase is extracted twice with DCM. The organic phases are combined, dried over anhydrous sodium sulfate, evaporated and purified by flash chromatography on silica gel (EtOAc/P.E.: 3/7+0.1% v/v of triethylamine) to give 1.3 g of a pale yellow oil (yield 85%).

¹H NMR 300 MHz (CDCl₃): δ (ppm)=1.45 (s, 9H, COOC(CH₃)₃); 2.90 (dd, J=4.9 Hz and J=17.6 Hz, 1H, CH₂COO); 3.10 (dd, J=4.9 Hz and J=17.7 Hz, 1H, CH₂COO); 4.40 (m, 1H, CαH); 5.15 (s, 2H, CH₂Ph); 5.62 (d, J=8.3 Hz, 1H, NH); 7.37 (m, 5H, aromatic); 9.70 (s, 1H, CHO).

IR (NaCl): ν_{NH}=3376 cm⁻¹; ν_{Csp2 Harom}=3066 cm⁻¹ and 3034 cm⁻¹; ν_{Csp3}=2978 cm⁻¹ and 2933 cm⁻¹; ν_{C═O}=1731 cm⁻¹; ν_{C═C}=1499 cm⁻¹ and 1455 cm⁻¹; ν_{C—O ester}=1253 cm⁻¹ and 1165 cm⁻¹.

[α]_{D}^{26.6° C.}=−3.88° (0.1 g/L, DCM).

g) 304 mg (0.99 mmol, 1.67 eq.) of aldehyde (V.I) and 216 mg (0.59 mmol, 1 eq.) of aza-amino acid (III.2) are dissolved in an MeOH/AcOH mixture: 99/1 (1 mL/40 mg of aldehyde). 1.33 eq. of sodium cyanoborohydride are added to this solution in 5-6 mg portions over 45 minutes. The medium is left stirring overnight at room temperature. The flask is then immersed in an ice bath and saturated NaHCO₃ solution (1 mL/10 mg of aldehyde) is added slowly. The aqueous phase is extracted three times with EtOAc. The organic phase is washed three times with saturated NaHCO₃ solution, dried over anhydrous sodium sulfate, evaporated and then purified by flash chromatography on silica gel (EtOAc/P.E.: 3/7 by volume) to give 265 mg of a yellow oil (yield 41%).

¹H NMR 300 MHz (CDCl₃): δ (ppm)=1.44 (s, 9H, COOC(CH₃)₃); 1.56-1.46 (m, 17H, CH₂—(CH₂)₄—CH₂ and COOC(CH₃)₃); 2.65 (m, 2H, CH₂COOBn); 2.95 (m, 2H, CH₂—NH); 3.18 (m, 2H, CH₂—N—N); 3.25 (m, 2H, CH₂—N—N); 4.00 (m, 1H, CHα); 4.30 (s, broad, 1H, NH); 4.85 (s, broad, 1H, NH); 5.12 (d, J=6.0 Hz, 4H, CH₂-Ph); 5.15 (m, 1H, NH); 7.45-7.30 (m, 10H, aromatic).

¹³C NMR 75 MHz (DMSO): δ (ppm)=26.23; 26.34; 28.41; 28.28; 29.89; 37.46; 40.98; 49.16; 53.47; 66.60; 66.50; 80.55; 79.55; 128.62; 128.53; 128.33; 128.28; 128.09; 136.74; 135.74; 155.38; 156.48; 156.30; 171.30.

IR (NaCl): ν_{NH}=3339 cm⁻¹; ν_{Csp2 Harom}=3065 cm⁻¹ and 3033 cm⁻¹; ν_{Csp3}=2976 cm⁻¹ and 2933 cm⁻¹; ν_{C═O}=1696 cm⁻¹; ν_{C═C}=1523 cm⁻¹ and 1455 cm⁻¹; ν_{C—O ester}=1248 cm⁻¹ and 1166 cm⁻¹.

[α]_{D}^{25° C.}=−0.61° (18.9 g/L, DCM)

HRMS-ESI (m/z): [M+Na] calc. for C₃₅H₅₂N₄O₈, 679.3683; exp., 679.3688.

### EXAMPLE 10

### Benzyl ester of (3S)-4-[2-(3-benzyloxycarbonylaminopropyl)-2-tert-butoxycarbonylhydrazino]-3-tert-butoxycarbonylamino-4-oxobutyric acid (IB.2)

### a)1-tert-Butoxycarbonyl-1-(3-benzyloxycarbonylaminopropyl)-hydrazine (III.5)

Compound (III.5) is obtained according to a procedure identical to that followed for the synthesis of (III.2) using tert-butoxycarbonylhydrazine and 3-(benzyloxycarbonylamino)propanol as starting materials and replacing the

DIAD with DEAD in the Mitsunobu reaction.

¹H NMR 300 MHz (CDCl₃): δ (ppm)=1.50 (s, 9H, COOC(CH₃)₃); 1.80 (quint, J=19.2 Hz, J=13.2 Hz and J=6.4 Hz, 2H, CH₂—CH₂—CH₂); 3.25 (q, J=6.4 Hz and J=12.8 Hz, 2H, CH₂NH); 3.50 (t, J=6.4 Hz, 2H, N—N—CH₂); 5.10 (s, 2H, COOCH₂-Ph); 6.50 (s, broad, 1H, NH); 7.40 (m, 5H, phenyl).

IR (NaCl): ν_{NH}=3335 cm⁻¹; ν_{Csp2 Harom}=3066 cm⁻¹ and 3035 cm⁻¹; ν_{Csp3}=2977 cm⁻¹ and 2934 cm⁻¹; ν_{C═O}=1694 cm⁻¹; ν_{C—O ester}=1250 cm⁻¹ and 1143 cm⁻¹.

b) To a solution of aspartic acid (20 mg, 0.17 mmol, 1.1 eq.) in DMF (2 mL/0.1 mmol of aspartic acid) are added 1.3 eq. of HOBt and then 1.3 eq. of EDCI. The aza-amino acid (III.5) (50 mg, 0.15 mmol, 1 eq.) is then added. The solution is stirred at room temperature overnight. The solution is diluted in ethyl acetate and then washed three times with saturated NaHCO₃ solution, and then with 1N HCl. The organic phase is dried over anhydrous sodium sulfate and then is evaporated. The residue is chromatographed on silica gel (MeOH/DCM: 1/99 by volume) to give 20 mg of a pale yellow oil (yield 20%).

¹H NMR 300 MHz (CDCl₃): δ (ppm)=1.40 (s, 9H, COOC(CH₃)₃); 1.45 (s, 9H, COOC(CH₃)₃); 1.70 (m, 2H, CH₂—CH₂—CH₂); 2.80 (dd, J=17.5 Hz and J=4.5 Hz, 1H, CH₂COOBn); 3.00 (dd, J=17.5 Hz and J=4.5 Hz, 1H, CH₂COOBn); 3.30 (m, 2H, CH₂NH); 3.50 (m, 2H, N—N—CH₂); 4.60 (m, 1H, CαH); 5.12 (s, 2H, CH₂Ph); 5.15 (s, 2H, CH₂Ph); 5.62 (d, 1H, NH); 7.40 (m, 10H, aromatic); 8.40 (s, 1H, CONHN).

¹³C NMR 75 MHz (CDCl₃): 27.96 (1C, CH₂—CH₂—CH₂—NHZ); 28.50 (3C, COOC(CH₃)₃); 28.69 (3C, COOC(CH₃)₃); 36.20 (1C, CH₂ asp); 38.39 (1C, CH₂—CH₂—CH₂NHZ); 47.00 (1C, N—N—CH₂—CH₂—CH₂—NHZ); 49.73 (1C, CαH); 66.94 (1C, CH₂-PH); 67.38 (1C, CH₂-PH); 77.04 (1C, COOC(CH₃)₃); 77.88 (1C, COOC(CH₃)₃); 128.39 (2C, CHar); 128.48 (2C, CHar); 128.68 (1C, CHar); 128.86 (1C, CHar); 129.02 (2C, CHar); 129.14 (2C, CHar); 135.67 (1C, C^{IV}ar); 137.12 (1C, C^{IV}ar); 153.25 (NHCOO); 155.98 (1C, NHCOO); 157.04 (1C, CH₂COO); 169.80 (1C, CONHN); 171.88 (1C, CONNH).

[α]_{D}^{22.6° C.}=+7.14° (2.8 g/L, DCM)

HRMS-ESI (m/z): [M+Na] calc. for C₃₂H₄₄N₄O₉, 651.3006; exp., 651.3010.

### EXAMPLE 11

### Benzyl ester of (3S)-4-[2-(4-benzyloxycarbonylaminophenyl)-2-tert-butoxycarbonylhydrazino]-3-tert-butoxycarbonylamino-4-oxobutyric acid (IB3)

### a) Benzyl ester of (4-iodophenyl)carbamic acid (5)

0.5 g of 4-iodoaniline (4) (2.28 mmol, 1 eq.) are dissolved in 20 mL of DCM. 1 mL of distilled triethylamine is added and the mixture is cooled to 0° C. A solution of benzyl chloroformate (1.95 mL, 13.7 mmol, 6 eq.) in 20 mL of DCM is added dropwise, with stirring. Once the addition is complete, the mixture is left for 15 minutes at 0° C. and then warmed to room temperature. The reaction medium is then refluxed for 20 hours. After cooling to room temperature, the reaction mixture is poured into 1M HCl solution. After dilution with DCM, the organic phase is recovered, washed with saturated Na₂CO₃ solution, dried over Na₂SO₄, filtered and evaporated to dryness. The crude product obtained is chromatographed on silica gel, eluting with a DCM/P.E. mixture (3/2 by volume). A white powder is obtained (m=460 mg) (yield 60%).

¹H NMR 300 MHz (DMSO): δ (ppm)=5.14 (s, 2H, CH₂—C₆H₅); 7.28-7.69 (m, 9H, Hₐᵣₒₘ); 9.89 (s, 1H, NH).

IR (KBr): ν_{NH}=3322 cm⁻¹; ν_{Csp2 Harom}=3034 cm⁻¹; ν_{C═O}=1701 cm⁻¹; ν_{C═C}=1528 cm⁻¹; ν_{C—O ester}=1236 cm⁻¹.

Melting point=130° C.

### b) tert-Butyl ester of 1-(4-benzyloxycarbonylaminophenyl)-hydrazinecarboxylic acid (III.1)

Distilled DMF (4.5 mL) is introduced into a round-bottomed flask. Four “freezing-vacuum-thawing” cycles are performed. After placing under an argon atmosphere, 1 g of compound (5) (2.83 mmol, 1 eq.), 54 mg of CuI (0.283 mmol, 0.1 eq.), 51 mg of 1,10-phenanthroline (0.283 mmol, 0.1 eq.), 1.29 g of Cs₂CO₃ (3.96 mmol, 1.4 eq.) and 450 mg of tert-butyl carbazate (3.39 mmol, 1.2 eq.) are introduced. A further four “freezing-vacuum-thawing” is cycles are performed, and the medium is placed under an argon atmosphere and heated to 80° C. The argon stream is stopped after 20 minutes. The reaction is left at 80° C. for 18 hours. After cooling to room temperature, the medium is filtered on a sinter through a bed of silica one centimeter thick, rinsing with ethyl acetate. The ethyl acetate and the DMF are evaporated off to give a crude product, which is chromatographed on silica gel with a P.E./EtOAc evolution gradient from (2/1) to (1/1) by volume. 100 mg of a straw-yellow powder are obtained (yield 10%).

¹H NMR 300 MHz (CDCl₃): δ (ppm)=1.43 (s, 9H, C(CH₃)₃); 5.00 (sl, 2H, NH₂); 5.14 (s, 2H, CH₂—C₆H₅); 7.29-7.55 (m, 9H, Hₐᵣₒₘ); 9.7 (s.1H, NH).

¹³C NMR 75 MHz (CDCl₃): δ (ppm)=28.63; 28.72; 28.76; 68.36; 71.73; 113.21; 113.3; 117.43; 122.06; 122.13; 129.63; 129.74; 129.98; 131.16; 138.04; 138.94; 153.54; 155.92.

IR (KBr): ν_{NH}=3323 cm⁻¹; ν_{Csp3}=2978 cm⁻¹; ν_{C═O}=1699 cm⁻¹ and 1603 cm⁻¹; ν_{C═C}=1538 cm⁻¹.

c) According to a procedure identical to that described for compound (IB.2), 100 mg of aza-amino acid (III.1) (0.27 mmol, 1 eq.) and 100 mg of Boc-Asp(OBn)-OH (0.31 mmol, 1.1 eq.) lead, after purification by flash chromatography on silica gel, eluting with an EtOAc/P.E. mixture (1/1 by volume), to 120 mg of a straw-yellow powder (yield 64%).

¹H NMR 300 MHz (DMSO): δ (ppm)=1.4 (s, 18H, C(CH₃)₃); 4.1 (m, 2H, CH₂β); 4.41 (m, 1H, CHα); 5.18 (s, 2H, CH₂—C₆H₅); 5.76 (s, 2H, CH₂—C₆H₅); 7.2-7.6 (m, 14H, Hₐᵣₒₘ); 9.8 (s, 1H, NH); 10.6 (s, 1H, NH).

¹³C NMR 75 MHz (CDCl₃): δ (ppm)=14.6; 21.46; 28.28; 28.50; 28.69; 28.75; 30.1; 36.24; 49.74; 60.84; 67.36; 77.10; 77.52; 77.95; 81.15; 82.73; 84.32; 119.19; 125.29; 128.69; 128.71; 128.79; 129.00; 135.73; 136.27; 136.50; 137.26; 150.52; 153.36; 153.82; 156.04; 169.54; 170.55; 171.66; 171.8.

IR (KBr): ν_{NH}=3347 cm⁻¹; ν_{Csp3}=2980 cm⁻¹; ν_{C═O}=1721 cm⁻¹, 1703 cm⁻¹ and 1676 cm⁻¹; ν_{C—O ester}=1162 cm⁻¹.

Melting point=109° C.

HRMS-ESI (m/z): [M+Na] calc. for C₃₅H₄₂N₄O₉, 685.2849; exp., 685.2854.

### EXAMPLE 12

### Benzyl ester of (4S)-5-[2-(4-benzyloxycarbonylaminophenyl)-2-tert-butoxycarbonylhydrazino]-4-tert-butoxycarbonylamino-5-oxopentanoic acid (IB.4)

According to a procedure identical to that described for compound (IB.2), 100 mg of aza-amino acid (MA) (0.27 mmol, 1 eq.) and 105 mg of Boc-Glu(OBn)-OH (0.31 mmol, 1.1 eq.) lead, after purification by flash chromatography on silica gel, eluting with an EtOAc/P.E. mixture (1/1 by volume), to 60 mg of a straw-yellow powder (yield 32%).

¹H NMR 300 MHz (CDCl₃): δ (ppm)=1.46 (s, 9H, C(CH₃)); 1.48 (s, 9H, C(CH₃)); 1.7 (m, 2H, CH₂); 2.6 (m, 2H, CH₂); 4.3 (m, 1H, CHα); 5.15 (s, 2H, CH₂—C₆H₅); 5.21 (s, 2H, CH₂—C₆H₅); 6.7 (s, 1H, NH); 7.3-7.5 (m, 14H, Hₐᵣₒₘ); 8.6 (s, 1H, NH).

¹³C NMR 75 MHz (CDCl₃): δ (ppm)=28.04; 28.53; 28.68; 30.11; 30.74; 52.26; 65.71; 67.03; 67.44; 77.03; 77.45; 77.65; 77.87; 80.86; 82.83; 119.21; 125.48; 127.4; 128.67; 128.73; 128.76; 129.02; 136.06; 136.23; 136.40; 137.40; 138.14; 153.43; 153.77; 156.26; 171.28; 173.55.

IR (KBr): ν_{NH}=3350 cm⁻¹; ν_{Csp3}=2981 cm⁻¹; ν_{C═O}=1703 cm⁻¹, 1724 cm⁻¹ and 1676 cm⁻¹; ν_{C═C}=1530 cm⁻¹; ν_{C—O ester}=1164 cm⁻¹.

Melting point=115° C.

HRMS-ESI (m/z): [M+Na] calc. for C₃₆H₄₄N₄O₉, 699.3006; exp., 699.3010.

### EXAMPLE 13

### Benzyl ester of (3S)-4-[2-(5-benzyloxycarbonylaminopentyl)-2-tert-butoxycarbonylhydrazino]-3- tert-butoxycarbonylamino-4-oxobutyric acid (IB.5)

### a) tert-Butyl ester of 1-(5-benzyloxycarbonylamnopentyl)-hydrazinecarboxylic acid (III.6)

Compound (III.6) is obtained according to a procedure identical to that followed for the synthesis of (III.2) using tert-butoxycarbonylhydrazine and 5-(benzyloxycarbonylamino)pentanol as starting materials and replacing the DIAD with DBAD in the Mitsunobu reaction.

¹H NMR 300 MHz (CDCl₃): δ (ppm)=1.40-1.70 m, 15H, CH₂—(CH₂)₃—CH₂ and COOC(CH₃)₃); 3.20 (q, J=6.8 Hz and J=6.4 Hz, 2H, CH₂—NHZ); 3.35 (t, J=7.2 Hz, 2H, CH₂—N—N); 4.00 (s, 2H, NH₂); 4.80 (s, broad, 1H, NHZ); 5.10 (s, 2H, CH₂Ph); 7.36 (m, 5H, aromatic).

IR (NaCl): μ_{NH}=3340 cm⁻¹; ν_{Csp2 Harom}=3065 cm⁻¹ and 3033 cm⁻¹; ν_{Csp3}=2975 cm⁻¹ and 2933 cm⁻¹; ν_{C═O}=1694 cm⁻¹; ν_{C—O ester}=1251 cm⁻¹ and 1143 cm⁻¹.

b) According to a procedure identical to that described for compound (IB.2), 120 mg of aza-amino acid (III.6) (0.34 mmol, 1 eq.) and 120 mg of Boc-Asp(OBn)-OH (0.37 mmol, 1.1 eq.) lead, after purification by flash chromatography on silica gel, eluting with an EtOAc/P.E. mixture (4:6 by volume), to 136 mg of a yellow oil (yield 61%).

¹H NMR 300 MHz (CDCl₃): δ (ppm)=1.10-1.30 (m, 24H, CH₂—(CH₂)₃—CH₂ and COOC(CH₃)₃); 2.66-2.94 (m, 2H, CH₂COOBn); 3.12 (m, 2H, CH₂—NHZ); 3.36 (t, J=6.4 Hz, 2H,CH₂—N—N); 4.50 (s, broad, 1H, CHα); 4.85 (s, broad, 1H, NH); 5.05 (d, J=12.8 Hz, 4H, CH₂-Ph); 5.60 (s, broad, 1H, NH); 7.30 (m, 10H, aromatic); 8.30 (s, 1H, CO—NH—N).

¹³C NMR 75 MHz (CDCl₃): δ (ppm)=26.19; 27.27; 28.20; 28.33; 29.73; 35.91; 40.83; 49.36; 66.60; 66.95; 80.76; 81.27; 128.08; 128.28; 128.44; 128.53; 128.64; 135.38; 136.75; 154.67; 155.59; 156.5; 171.51.

IR (NaCl): ν_{NH}=3323 cm⁻¹; ν_{Csp2 Harom}=3034 cm⁻¹; ν_{Csp3}=2978 cm⁻¹ and 2934 cm⁻¹; ν_{C═O}=1694 cm⁻¹; ν_{C—O ester}=1250 cm⁻¹ and 1161 cm⁻¹.

[α]_{D}^{24.3° C.}=−13° (10 g/L, DCM)

HRMS-ESI (m/z): [M+Na] calc. for C₃₄H₄₈N₄O₉, 679.3319; exp., 679.3329.

### EXAMPLE 14

### Benzyl ester of 4-[2-((2S)-3-benzyloxycarbonyl-2-tert-butoxycarbonylaminopropyl)-1-tert-butoxycarbonylhydrazino-methyl]piperidine-1-carboxylic acid (IB.6)

### a) Benzyl ester of 4-(1-tert-Butoxycarbonylhydrazinomethyl)-piperidine-1-carboxylic acid (III.7)

Compound (III.7) is obtained according to a procedure identical to that followed for the synthesis of (III.2) using tert-butoxycarbonylhydrazine and the benzyl ester of 4-hydroxymethylpiperidine-1-carboxylic acid as starting materials and replacing the DIAD with DBAD in the Mitsunobu reaction.

¹H NMR 300 MHz (CDCl₃): δ (ppm)=1.38 (s, 9H, COOC(CH₃)₃); 1.50-1.65 (m, 4H, CH₂—CH₂—N); 1.82 (m, 1H, CHα); 2.70 (m, 2H, CH₂—N—NH₂); 3.18 (d, J=7.2 Hz, 2H, CH₂—CH₂—N); 3.90 (s, 2H, NH₂); 4.10 (s, 2H, CH₂—CH₂—N); 5.05 (s, 2H, CH₂Ph); 7.30 (m, 5H, aromatic).

IR (NaCl): ν_{NH}=3500 cm⁻¹ and 3334 cm⁻¹; ν_{Csp2 Harom}=3065 cm⁻¹; νCsp3=2974 cm⁻¹ and 2927 cm⁻¹; ν_{C═O}=1694 cm⁻¹ and 1682 cm⁻¹; ν_{C—O ester}=1278 cm⁻¹ and 1220 cm⁻¹.

b) According to a procedure identical to that described for compound (IB.1), 116 mg of aza-amino acid (III.7) (0.34 mmol, 1 eq.) and 162 mg of compound (V.1) (0.53 mmol, 1.5 eq.) lead, after purification by flash chromatography on silica gel, eluting with an EtOAc/cyclohexane mixture (3/7 by volume), to 167 mg of a yellow oil (yield 80%).

¹H NMR 300 MHz (CDCl₃): δ (ppm)=1.30-1.40 (m, 18H, COOC(CH₃)₃); 1.50-1.70 (m, 4H, CH₂—CH₂—N); 1.78 (m, 1H, CHα-; 2.55 (d, J=5.7 Hz, 2H, CH₂—COOBn); 2.70 (m, 2H, CH₂—NH—N); 2.90 (m, 2H, CH₂—N—NH); 3.10 (m, 2H, CH₂—CH—N); 3.90 (s, broad, 1H, NH); 4.08 (m, 2H, CH₂—CH₂—N); 5.05 (m, 4H, CH₂Ph); 5.10 (m, 1H, NH); 7.28 (m, 10H, aromatic).

¹³C NMR 75 MHz (CDCl₃): δ (ppm 26.95; 28.27; 28.42; 29.68; 34.73; 37.47; 43.89; 46.70; 53.25; 54.34; 66.57; 67.07; 80.89; 127.89; 128.31; 128.40; 128.52; 128.65; 135.67; 136.93; 155.34; 155.41; 170.63; 171.25.

IR (NaCl): ν_{NH}=3334 cm⁻¹; ν_{Csp2 Harom}=3091 cm⁻¹ and 3033 cm⁻¹; ν_{Csp3}=2976 cm⁻¹ and 2929 cm⁻¹; ν_{C═O}=1695 cm⁻¹; ν_{C—O ester}=1278 cm⁻¹ and 1249 cm⁻¹.

[α]_{D}²⁵° C.=−0.80° (7.1 g/L, DCM).

HRMS-ESI (m/z): [M+Na] calc. for C₃₅H₅₀N₄O₈, 677.3526; exp., 677.3532.

### EXAMPLE 15

### Benzyl ester of 4-[2-((2S)-3-benzyloxycarbonyl-2-tert-butoxycarbonylaminopropionyl)-1-tert-butoxycarbonylhydrazino-methyl]piperidine-1-carboxylic acid (IB.7)

According to a procedure identical to that described for compound (IB.2), 145 mg of aza-amino acid (III.7) (0.45 mmol, 1 eq.) and 160 mg of Boc-Asp(OBn)-OH (0.49 mmol, 1.1 eq.) lead, after purification by flash chromatography on silica gel, eluting with an EtOAc/cyclohexane mixture (3/7 by volume), to 210 mg of a yellow oil (yield 77%).

¹H NMR 300 MHz (CDCl₃): δ (ppm)=1.45 (m, 18H, COOC(CH₃)₃); 1.60-1.80 (m, 5H, CHα and CH₂—CH₂—N); 2.70-3.05 (m, 4H, CH₂—COOBn and CH₂—N—NH); 3.35 (m, 2H, CH₂—CH₂—N); 4.20 (m, 2H, CH₂—CH₂—N); 4.55 (s, broad, 1H, CHα); 5.15 (d, J=6.4 Hz, 4H, CH₂Ph); 5.68 (s, broad, 1H, NH); 7.38 (m, 10H, aromatic); 8.30 (s, 1H, CONHN).

¹³C NMR 75 MHz (CDCl₃): δ (ppm)=26.96; 28.18; 28.34; 29.69; 34.85; 43.85; 49.39; 55.00; 67.04; 80.87; 81.57; 127.88; 127.97; 128.26; 128.51; 128.66; 135.34; 136.96; 155.29; 155.66; 171.54.

IR (NaCl): ν_{NH}=3306 cm⁻¹; ν_{Csp2 Harom}=3034 cm⁻¹; ν_{Csp3}=2922 cm⁻¹ and 2853 cm⁻¹; ν_{C═O}=1701 cm⁻¹.

[α]_{D}²⁵° C.=−19.31° (14.1 g/L, DCM)

HRMS-ESI (m/z): [M+Na] calc. for C₃₅H₄₈N₄O₉, 691.3319; exp., 691.3322.

### EXAMPLE 16

### Benzyl ester of (3S)-4-[2-(5-benzyloxycarbonylaminopentyl)-2-tert-butoxycarbonylhydrazino]-3-tert-butoxycarbonylaminobutyric acid (IB.8)

According to a procedure identical to that described for compound (IB.1), 192 mg of aza-amino acid (III.6) (0.54 mmol, 1 eq.) and 280 mg of compound (V.1) (0.91 mmol, 1.6 eq.) lead, after purification by flash chromatography on silica gel, eluting with an EtOAc/P.E. mixture (3/7 by volume), to 295 mg of a yellow oil (yield 85%).

¹H NMR 300 MHz (CDCl₃): δ (ppm)=1.32-1.40 (m, 18H, COOC(CH₃)₃); 1.41-1.52 (m, 6H, CH₂—(CH₂)₃—CH₂); 2.60 (m, 2H, CH₂COOBn); 2.95 (m, 2H, CH₂—NH); 3.12 (m, 2H, CH₂—N—N); 3.25 (m, 2H,CH₂N—N); 3.95 (m, 1H, CHα); 4.65 (s, broad, 1H, NH); 4.80 (s, broad, 1H, NH); 5.05 (d, J=7.9 Hz, 4H, CH₂-Ph); 5.15 (s, 1H, broad, NH); 7.20-7.35 (m, 10H, aromatic).

¹³C NMR 75 MHz (CDCl₃): δ (ppm)=23.77; 28.29; 28.41; 28.45; 29.57; 37.42; 40.98; 49.08; 53.46; 66.51; 66.62; 79.56; 80.63; 128.10; 128.20; 128.29; 128.34; 128.53; 128.62; 135.74; 136.71; 155.38; 156.50; 171.14; 171.31.

IR (NaCl): ν_{NH}=3344 cm⁻¹; ν_{Csp2 Harom}=3065 cm⁻¹ and 3034 cm⁻¹; ν_{Csp3}=2977 cm⁻¹ and 2934 cm⁻¹; ν_{C═O}=1694 cm⁻¹; ν_{C═C}=1520 cm⁻¹ and 1455 cm¹; ν_{C—O ester}=1250 cm⁻¹ and 1167 cm⁻¹.

[α]_{D}^{25° C.}=−1.04° (18.2 g/L, DCM).

HRMS-ESI (m/z): [M+Na] calc. for C₃₄H₅₀N₄O₈, 665.3526; exp., 665.3527.

### EXAMPLE 17

### Benzyl ester of (3S)-4-[2-(6-benzyloxycarbonylaminohexyl)-2-tert-butoxycarbonylhydrazino]-3-tert-butoxycarbonylamino-4-oxobutyric acid (IB.9)

According to a procedure identical to that described for compound (IB.2), 120 mg of aza-amino acid (III.2) (0.55 mmol, 1 eq.) and 196 mg of Boc-Asp(OBn)-OH (0.60 mmol, 1.1 eq.) lead, after purification by flash chromatography on silica gel, eluting with an EtOAc/P.E. mixture (4:6 by volume), to 297 mg of a yellow oil (yield 80%).

¹H NMR 300 MHz (CDCl₃): δ (ppm)=1.15-1.30 (m, 26H, CH₂—(CH₂)₄—CH₂ and COOC(CH₃)₃); 2.60-2.96 (m, 2H, CH₂COOBn); 3.10 (m, 2H, CH₂—NHZ); 3.38 (m, 2H,CH₂—N—N); 4.50 (s, broad, 1H, CHα); 4.80 (s, broad, 1H, NH); 5.05 (d, 4H, CH2Ph); 5.65 (s, broad, 1H, NH); 7.30 (m, 10H, aromatic); 8.20 (s, 1H, CONHN).

¹³C NMR 75 MHz (CDCl₃): δ (ppm)=23.43; 26.83; 28.20; 28.33; 29.44; 40.48; 49.44; 66.65; 66.95; 80.73; 81.31; 128.07; 128.13; 128.29; 128.43; 128.52; 128.64; 135.39; 136.71; 154.71; 156.63; 171.47.

IR (NaCl): ν_{NH}=3320 cm⁻¹; ν_{Csp2 Harom}=3034 cm⁻¹; ν_{Csp3}=2977 cm⁻¹ and 2933 cm⁻¹; ν_{C═O}=1691 cm⁻¹; ν_{C═C}=1524 cm⁻¹ and 1455 cm⁻¹; ν_{C—O ester}=1250 cm⁻¹ and 1159 cm⁻¹.

[α]_{D}^{24.9}° C.=−10° (10 g/L, DCM).

HRMS-ESI (m/z): [M+Na] calc. for C₃₅H₅₀N₄O₉, 693.3475; exp., 693.3669.

### EXAMPLE 18

### Benzyl ester of (3S)-3-tert-butoxycarbonylamino-4-[2-tert-butoxycarbonyl-2-(6-tert-butoxycarbonylaminohexyl)hydrazino]-butyric acid (IB.10)

### a) 1-tert-Butoxycarbonyl-1-(6-tert-butoxycarbonylaminohexyl)-hydrazine (III.8)

Compound (III.8) is obtained according to a procedure identical to that followed for the synthesis of (III.2) using tert-butoxycarbonylhydrazine and 6-(Boc-amino)hexanol as starting materials and replacing the DIAD with DBAD in the Mitsunobu reaction.

¹H NMR 300 MHz (CDCl₃): δ (ppm)=1.36 (s, 9H, COO(CH₃)₃); 1.39 (s, 9H, COO(CH₃)₃); 1.19-1.48 (m, 8H, CH₂—(CH,)₄—CH₂); 2.89 (q, 2H, CH₂—NHBoc); 3.21 (t, 2H, CH₂—N—NH₂); 4.38 (s, 2H, NH₂); 6.76 (s, 1H, NH).

IR (KBr): ν_{NH}=3342 cm⁻¹; ν_{Csp3}=2976 cm⁻¹ and 2932 cm⁻¹; ν_{C═O}=1690 cm⁻¹; ν_{C═C}=1525 cm⁻¹; ν_{C—O ester}=1250 cm⁻¹ and 1170 cm⁻¹.

b) According to a procedure identical to that described for compound (IB.1), 130 mg of aza-amino acid (III.8) (0.4 mmol, 1 eq.) and 200 mg of compound (V.1) (0.7 mmol, 1.67 eq.) lead, after purification by flash chromatography on silica gel, eluting with an EtOAc/cyclohexane mixture (3/7 by volume), to 190 mg of a colorless oil.

¹H NMR 300 MHz (CDCl₃): δ (ppm)=1.35 (s, 9H, COO(CH₃)₃); 1.38 (s, 9H, COO(CH₃)₃); 1.39 (s, 9H, COO(CH₃)₃); 1.23-1.45 (m, 8H, CH₂—(CH₂)₄—CH₂); 2.63-2.83 (m, 4H, CH₂—NHZ and CH₂COOBn); 2.88 (m, 2H, CH₂—NH—N); 3.18 (m, 2H, CH₂—N—NH); 3.87 (m, 1H, CHα); 5.05 (s, 2H, CH₂Ph); 6.75 (m, 1H, NH); 7.36-7.30 (m, 5H, Hₐᵣₒₘ).

IR (NaCl): ν_{NH}=3353 cm⁻¹; σ_{Csp2 Harom}=3065 cm⁻¹ and 3027 cm⁻¹; ν_{Csp3}=2977 cm⁻¹ and 2932 cm⁻¹; ν_{C═O}=1694 cm⁻¹; ν_{C—O ester}=1250 cm⁻¹ and 1167 cm⁻¹.

HRMS-ESI (m/z): [M+Na] calc. for C₃₂H₅₄N₄O₈, 645.3839; exp., 645.3834.

### EXAMPLE 19

### Benzyl ester of (3S)-4-[2-benzyloxycarbonyl-2-(6-benzyloxycarbonylaminohexyl)hydrazino]-3-tert-butoxycarbonyl-aminobutyric acid (IB.11)

According to a procedure identical to that described for compound (IB.1), 130 mg of aza-amino acid (III3) (0.4 mmol, 1 eq.) and 200 mg of compound (V.1) (0.7 mmol, 1.67 eq.) lead, after purification by flash chromatography on silica gel, eluting with an EtOAc/cyclohexane mixture (3/7 by volume), to 120 mg of a white solid.

¹H NMR 300 MHz (CDCl₃): δ (ppm)=1.35 (s, 9H, COO(CH₃)₃); 1.30-1.46 (m, 8H, CH₂—(CH₂)₄—CH₂); 2.52-2.83 (m, 4H, CH₂—NHZ and CH₂COOBn); 2.94 (m, 2H, CH₂—NH—N); 3.25 (m, 2H, CH₂—N—NH); 3.87 (m, 1H, CHα); 4.99 (s, 2H, CH₂-Ph); 5.04 (s, 2H, CH₂-Ph); 5.06 (s, 2H, CH₂Ph); 7.20-7.38 (m, 15H, Hₐᵣₒₘ).

HRMS-ESI (m/z): [M+Na] calc. for C₃₈H₅₀N₄O₉, 713.3526; exp., 713.3520.

### EXAMPLE 20

### Benzyl ester of (3S)-3-benzyloxycarbonylamino-4-[2-benzyloxycarbonyl-2-(6-benzyloxycarbonylaminohexyl)hydrazino]-butyric acid (IB.12)

### a) Benzyl ester of 3-benzyloxycarbonylamino-4-oxobutanoic acid (V.2)

Compound (V.2) is obtained according to a procedure identical to that followed for the synthesis of (V.1) using Z-Asp(OBn)-OH as starting material.

IR (NaCl): ν_{NH}=3354 cm⁻¹; ν_{Csp2 Harom}=3065 cm⁻¹ and 3034 cm⁻¹; ν_{Csp3}=2931 cm⁻¹; ν_{C═O}=1731 cm⁻¹, 1715 cm⁻¹ and 1693 cm⁻¹; ν_{C═C}=1528 cm⁻¹; ν_{C—O ester}=1259 cm⁻¹.

b) According to a procedure identical to that described for compound (IB.1), 28 mg of aza-amino acid (III.3) (0.07 mmol, 1 eq.) and 45 mg of compound (V.2) (0.13 mmol, 1.67 eq.) lead, after purification by flash chromatography on silica gel, eluting with an EtOAc/cyclohexane mixture (3/7 by volume), to 20 mg of a yellow oil.

¹H NMR 300 MHz (CDCl₃): δ (ppm)=1.10-1.46 (m, 8H, CH₂—(CH₂)_{4—CH}₂); 2.60-2.70 (m, 4H, CH₂—NHZ and CH₂COOBn); 2.94 (m, 2H, CH₂—NH—N); 3.25 (m, 2H, CH₂—N—NH); 3.95 (m, 1H, CHα); 4.96-5.12 (s, 8H, 4×CH₂—Ph); 7.20-7.40 (m, 20H, Hₐᵣₒₘ).

IR (NaCl): ν_{NH}=3333 cm⁻¹; ν_{Csp2 Harom}=3065 cm⁻¹ and 3034 cm⁻¹; ν_{Csp3}=2932 cm⁻¹ and 2857 cm⁻¹; ν_{C═O}=1695 cm⁻¹; ν_{C═C}=1531 cm⁻¹; ν_{C—O ester}=1254 cm⁻¹ and 1174 cm⁻¹.

[α]_{D}^{25° C.}=−0.06° (5 g/L, DCM).

HRMS-ESI (m/z): [M+Na] calc. for C₄₀H₄₈N₄O₆, 747.3370; exp., 747.3369.

### EXAMPLE 21

### Benzyl ester of (3S)-4-[2-benzyloxycarbonyl-2-(6-tert-butoxycarbonylaminohexyl)hydrazino]-3-tert-butoxycarbonyl-aminobutyric acid (IB.13)

According to a procedure identical to that described for compound (IB.1), 153 mg of aza-amino acid (III.4) (0.4 mmol, 1 eq.) and 200 mg of compound (V.1) (0.7 mmol, 1.67 eq.) lead, after purification by flash chromatography on silica gel, eluting with an EtOAc/cyclohexane mixture (3/7 by volume), to 130 mg of a yellow oil.

¹H NMR 300 MHz (DMSO): δ (ppm)=1.35 (s, 18H, 2×COO(CH₃)₃); 1.29-1.49 (m, 8H, CH₂—(CH₂)₄—CH₂); 2.41-2.86 (m, 4H, CH₂—NHBoc and CH₂COOBn); 2.88 (m, 2H, CH₂—NH—N); 3.25 (m, 2H, CH₂—N—NH); 3.86 (m, 1H, CHα); 5.05 (s, 2H, CH₂-Ph); 5.07 (s, 2H, CH₂-Ph); 7.29-7.36 (m, 10H, Hₐᵣₒₘ).

HRMS-ESI (m/z): [M+Na] calc. for C₃₈H_{50 N}₄O₉, 679.3683; exp., 679.3659.

### EXAMPLE 22

### tert-Butyl ester of (2S)-6-(benzyloxycarbonylamino)-2-[1-(benzyloxycarbonylmethyl)-2-(tert-butoxycarbonyl)hydrazino-carbonylamino]hexanoic acid (IB.14)

### a) tert-Butyl ester of 2-isopropylidene-hydrazinecarboxylic acid (2)

To a solution of Boc-hydrazine (4 g, 30.3 mmol, 1 eq.) in 55 mL of acetone are added 1.35 g of anhydrous MgSO₄ and 4 drops of acetic acid. The mixture is refluxed for 2 hours. The magnesium sulfate is filtered off and the solvent is then evaporated off to give 4.96 g of a white solid (yield 95%).

¹H NMR 300 MHz (CDCl₃): δ (ppm)=1.53 (s, 9H, COOC(CH₃)₃); 1.80 (s, 3H, CH₃); 2.05 (s, 3H, CH₃); 7.37 (s, 1H, NH).

IR (KBr): ν_{NH}=3348 cm⁻¹; ν_{Csp3}=2981 cm⁻¹ and 2920 cm⁻¹; ν_{C═O}=1725 cm⁻¹; ν_{C═N}=1648 cm⁻¹.

Melting point: 87° C.

### b) Benzyl (1-tert-butoxycarbonyl-2-isopropylidenehydrazino)-acetate (3)

4.9 g of compound (2) (28.5 mmol, 1 eq.) are dissolved in 60 mL of anhydrous DMF, under N₂. 1.4 g of NaH (60% in mineral oil, 34 mmol, 1.2 eq.) are introduced portionwise. Evolution of gas and setting to a solid are observed. In 60 mL of anhydrous DMF and then 5 mL of benzyl bromoacetate (31.4 mmol, 1.1 eq.) are added slowly. The solution turns dark orange. After one hour, the excess NaH is hydrolyzed by slow addition of water. The solution is extracted with twice 90 mL of EtOAc. The organic phases are washed with 90 mL of water and then 90 mL of saturated NaCl solution, dried over anhydrous Na₂SO₄ and then evaporated. The residue is chromatographed on silica gel, eluting with an EtOAc/P.E. mixture (1/2 by volume), to give 8.4 g of a yellow oil (yield 92%).

¹H NMR 300 MHz (CDCl₃): δ (ppm)=1.45 (s, 9H, COOC(CH₃)₃); 1.95 (s, 3H, CH₃); 2.05 (s, 3H, CH₃); 4.35 (s, 2H, N—CH₂); 5.20 (s, 2H, CH₂); 7.37 (m, 5H, Hₐᵣₒₘ).

IR (NaCl): ν_{Csp2 Harom}=3066 cm⁻¹ and 3034 cm⁻¹; ν_{Csp3}=2977 cm⁻¹ and 2932 cm⁻¹; ν_{C═O}=1754 cm⁻¹ and 1710 cm⁻¹.

### c) Benzyl hydrazinoacetate ditosylate (VIB.1)

To a solution of 1 g of compound (3) (3.13 mmol, 1 eq.) in 10 mL of ethanol and 100 μL of water are added 1.19 g of para-toluenesulfonic acid (6.25 mmol, 2 eq.). The solution is refluxed for 5 hours. The solvent is evaporated off to give 1.64 g of a pink-colored gel, which is used without further purification (yield 100%).

¹H NMR 300 MHz (CDCl₃): δ (ppm)=2.45 (s, 6H, 2 PhCH₃); ν_{C═O}=1724 cm⁻¹; 2.45 (m, 2H, N—CH₂); 5.10 (s, 2H, CH₂Ph); 7.10 (d, J=8.3 Hz, 4H, Hₐᵣₒₘ APTS); 7.40 (m, 5H, Hₐᵣₒₘ benzyl); 7.70 (d, J=8.3 Hz, 4H, Hₐᵣₒₘ APTS); 8.60 (bs, 5H, NH₂⁺ and NH₃⁺).

IR (NaCl): ν_{NH}=3337 cm⁻¹; ν_{Csp2 Harom}=3066 cm⁻¹ and 3034 cm⁻¹; ν_{Csp3}=2978 cm⁻¹ and 2935 cm⁻¹; ν_{C═O}=1724 cm⁻¹; ν_{S═O}=1052 cm⁻¹.

### d) Benzyl (2-tert-butoxycarbonylhydrazino)acetate (IVB.1)

To a solution of 330 mg of benzyl hydrazinoacetate ditosylate (0.63 mmol, 1 eq.) in 7 mL of an EtOH/H₂O (1/1 by volume) at 0° C. are added 139 μL of NMM (1.26 mmol, 2 eq.). 151 mg of (Boc)₂O (0.69 mmol, 1.1 eq.) are introduced. The solution is stirred at 0° C. for 15 minutes and then at room temperature for two hours. The organic solution obtained after adding ethyl ether is washed with saturated KH₂PO₄ solution. The aqueous phase is re-extracted with ether, and the combined organic phases are dried over anhydrous Na₂SO₄ and then evaporated off to give 128 mg of a yellow liquid (yield 66%).

¹H NMR 300 MHz (CDCl₃): δ (ppm)=1.35 (s, 9H, COOC(CH₃)₃); 4.10 (s, 2H, N—CH₂); 5.10 (s, 2H, CH₂Ph); 7.20 (m, 5H, Hₐᵣₒₘ).

IR (NaCl): ν_{NH}=3349 cm⁻¹; ν_{Csp2 Harom}=3070 cm⁻¹ and 3035 cm⁻¹; ν_{Csp3}=2980 cm⁻¹ and 2935 cm⁻¹; ν_{C═O}=1749 cm⁻¹ and 1708 cm⁻¹.

e) According to a procedure identical to that described for compound (IA.4), 105 mg of hydrazinoester (IVB.1) (0.37 mmol, 1 eq.) and 155 mg of HCl.H-Lys(Z)-OtBu (0.37 mmol, 1 eq.) lead, after purification by flash chromatography on silica gel, eluting with an EtOAc/P.E. mixture (1/2 by volume), to 70 mg of a yellow oil (yield 30%).

¹H NMR 300 MHz (CDCl₃): δ (ppm)=1.20 (m, 2H, CH₂—CH₂—CH₂—CH₂NHZ); 1.35 (s, 9H, COOC(CH₃)₃); 1.40 (s, 9H, COOC(CH₃)₃); 1.50-1.60 (m, 4H, CH₂—CH₂—CH₂—CH₂NHZ); 3.10 (q, J=7.5 Hz and J=14.3 Hz, 2H, CH₂NHZ); 4.10 (s, 2H, N—N—CH₂); 4.30 (m, 1H, CHα); 5.00 (s, 2H, CH₂Ph); 5.15 (s, 2H, CH₂Ph); 6.30 (bs, 1H, NH); 6.60 (bs, 1H, NH); 7.35 (m, 10H, Hₐᵣₒₘ).

HRMS-ESI (m/z): [M+Na] calc. for C₃₃H₄₆N₄O₉, 665.3162; exp., 665.3168.

### II. BIOLOGICAL ACTIVITY

This section relating to the biological activity of the compounds makes reference to the attached figures.

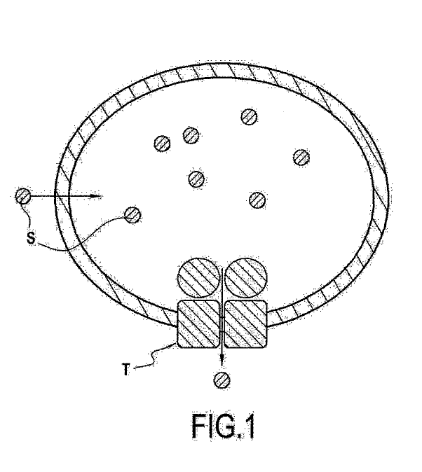

FIG. 1 is a scheme of efflux of substrate induced by a typical ABC transporter.

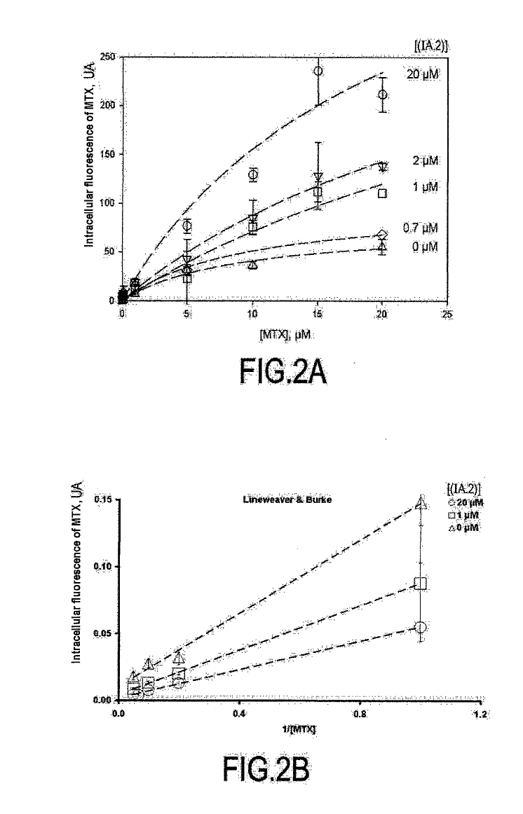

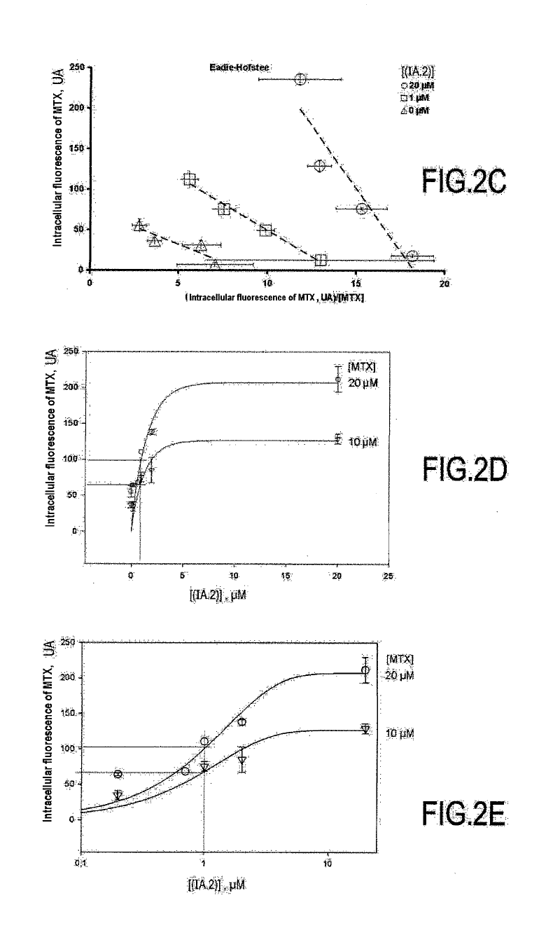

FIG. 2 demonstrates the type of inhibition of compound (I.A.2).

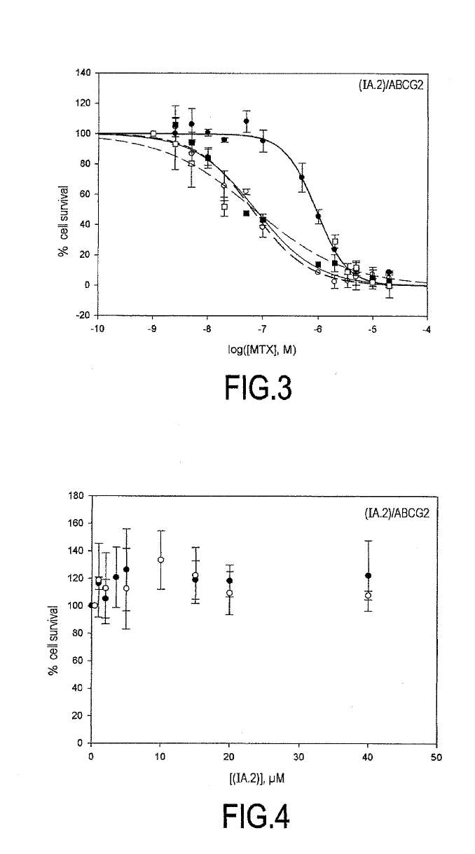

FIG. 3 shows the chemosensitization by compound (IA.2) of HEK-293 cells expressing ABCG2.

FIG. 4 shows the harmlessness of compound (IA.2) on HEK-293 cells.

FIG. 5 shows the chemosensitization by compound (IA.1) of HEK-293 cells expressing ABCG2.

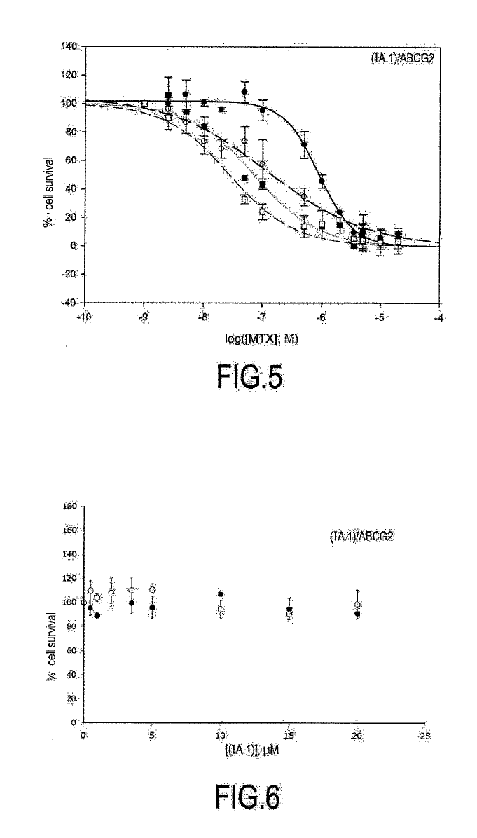

FIG. 6 shows the harmlessness of (IA.1) on HEK-293 cells.

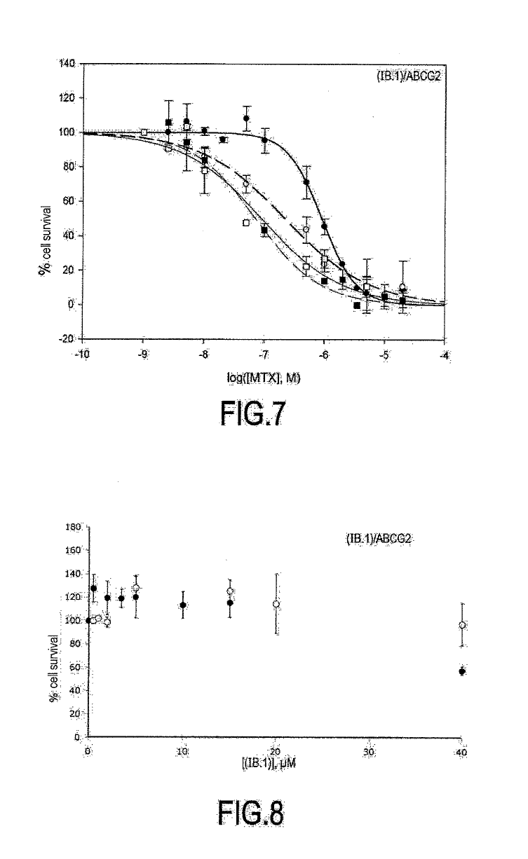

FIG. 7 shows the chemosensitization by compound (IB.1) of HEK-293 cells.

FIG. 8 shows the harmlessness of compound (IB.1) on HEK-293 cells.

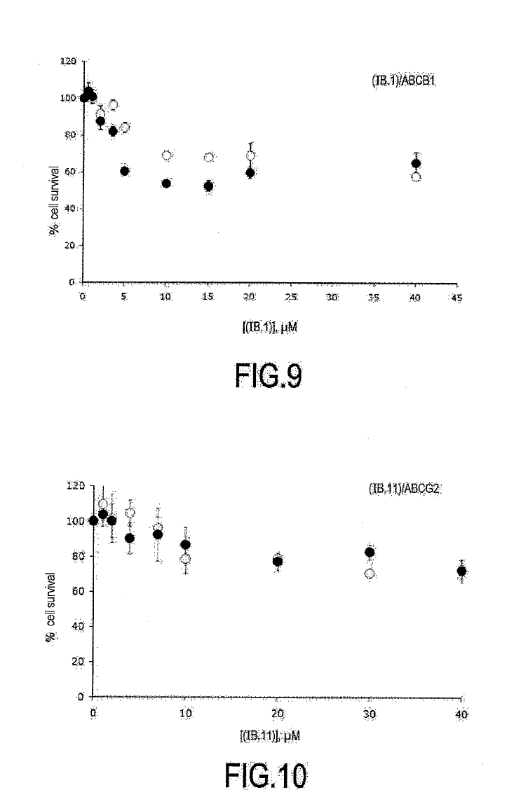

FIG. 9 shows the harmlessness of compound (IB.1) on NIH-3T3 cells.

FIG. 10 shows the harmlessness of compound (18.11) on HEK293 cells.

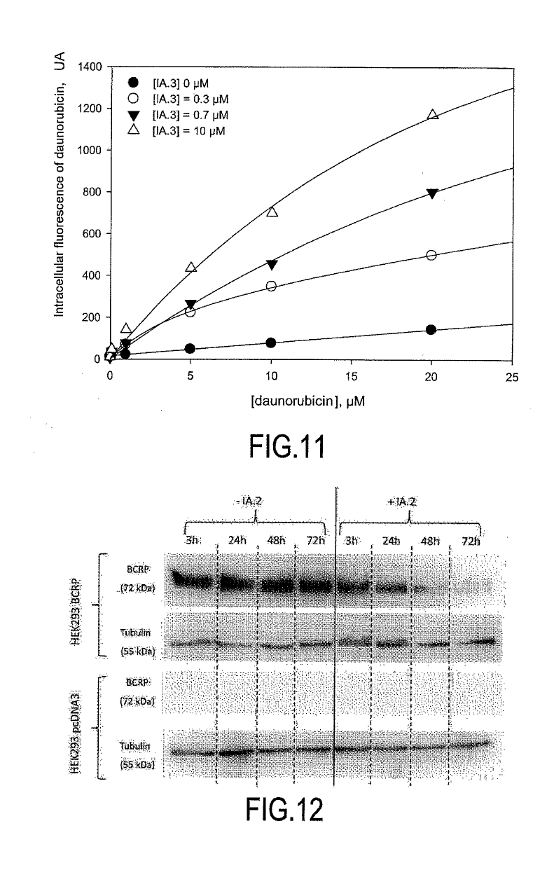

FIG. 11 demonstrates the type of inhibition of compound (I.A.3).

FIG. 12 shows that compound (IA.2) reduces the level of expression of ABCG2.

### METHODS

Cell cultures. The human fibroblast line HEK-293 transfected with the vector pcDNA3.1 including or not the gene encoding ABCG2 (Robey, R. W., Honjo, Y., Morisaki, K., Nadjem, T. A., Runge, S., Risbood, M., Poruchynsky, M. S., Bates, S. E. 2003, Br. J. Cancer 89(10) 1971-1978) was used to test the efficacy of the products on ABCG2. The newborn hamster kidney cell line BHK21 either natural (negative control) or transfected with the vector pNUT-MRP/His expressing the gene encoding ABCC1 (Chang, Xiu-Bao, Hou, Yue-Xian, Riordan, John R. 1997, J. Biol. Chem. 272(49) 30962-30968) was used to test the efficacy of the products on ABCC1. The cell line NIH3T3 natural or transfected with the vector pHaMDR1/A expressing ABCB1 (Cardarelli, Carol O., Aksentijevich, Ivan, Pastan, Ira, Gottesman, Michael M. 1995 Cancer Res. 55 1086-1091) was used to test the efficacy of the compounds on ABCB1. The various cell types are cultured according to the protocols described previously (Robey, R. W., Honjo, Y., Morisaki, K., Nadjem, T. A., Runge, S., Risbood, M., Poruchynsky, M. S., Bates, S. E. 2003, Br. J. Cancer 89(10) 1971-1978 for the line HEK293, Chang, Xiu-Bao, Hou, Yue-Xian, Riordan, John R. 1997, J. Biol. Chem. 272(49) 30962-30968 for the line BHK21 and Cardarelli, Carol O., Aksentijevich, Ivan, Pastan, Ira, Gottesman, Michael M. 1995 Cancer Res. 55 1086-1091 for the line NIH3T3).

Estimation of the level of expression of ABCG2. The cells HEK293 pcDNA3.1 or HEK293 pcDNA3.1 BCRP are seeded in sixteen 6 cm² Petri dishes and incubated for 24 hours at 37° C. under 5% CO₂. After this incubation, the various wells are supplemented with 30 μL either of DMSO (controls) or of compound IA.2 dissolved in DMSO, corresponding to a final concentration after dilution of 5 μM. After 3 h, 24 h, 48 h or 72 h of incubation, the cells are trypsinized and centrifuged at 200 g for 5 minutes. The protein fraction is extracted with a hypotonic lysis buffer (10 mM Tris-Cl pH 7.5, 10 mM NaCl, 1 mM MgCl₂, 1 mM DTT, protease inhibitors) and assayed with bicinchoninic acid (Sigma-Aldrich). Ten micrograms of this fraction are deposited on polyacrylamide gel of Laemmli type (U. K. Laemmli, Nature 1970, 227, 680) to separate the proteins, which are then transferred onto polyvinyl fluoride (pvdf) membrane for 60 minutes at 100 mA in a 10 mM CAPS pH 11.1 lysis buffer, 10% methanol (Sigma-Aldrich). The pvdf membrane is then saturated for 30 minutes in a solution of powdered skimmed milk at 1% (w/v) in TBST buffer (Tris buffer saline 1×, Tween 20 0.1%). After rapid rinsing with TBST, the membrane is incubated for 1 hour, with stirring at room temperature, with an anti-BCRP BXP-21 monoclonal antibody (Santa Cruz) or an anti-tubulin monoclonal antibody (Santa Cruz) diluted to 1/250 in 1% TBST milk. After 3 washes of 15 minutes with TBST, the membrane is incubated for 1 hour with a peroxidase-coupled anti-mouse secondary monoclonal antibody (Santa Cruz) diluted to 1/5000 in 1% TBST milk, and the membrane is then washed for three times 15 minutes in TBST. Revelation is performed using the “immobilon western” kit (Millipore) with the QuantityOne software (Biorad).

FIG. 1 schematically illustrates an efflux of substrate induced by a typical ABC transporter. The substrates S, such as mitoxantrone effluxed with ABCG2 and ABCB1 or daunorubicin effluxed with ABCC1, are accumulated in the cells and expelled by the transporter T, leading to a decrease in intracellular fluorescence. In the case of accumulation of the product, typically by inhibition of the efflux protein, the intracellular fluorescence increases.

Flow cytometry. Transfected or untransfected HEK-293 and BHK-21 cells are in a first stage placed in contact with substrate (5 μM mitoxantrone or 1 μM daunorubicin depending on the transporter). The substrate is accumulated for 30 minutes at 37° C. in the presence or absence of varied concentrations of the test molecules, at 0.5% of DMSO final. After washing with PBS, the cells are incubated in medium containing the same concentrations of test molecules as previously, for 1 hour at 37° C. The drug intracellular fluorescence is measured with a FACscan flow cytometer (Becton Dickinson, Mountain View, Calif.). The fluorescent substrates are excited at 488 nm; the fluorescence emission of mitoxantrone (MTX) is measured at 650 nm and that of daunorubicin is measured at 530 nm. The efficacy of the test molecules is estimated by means of equation 1:

% efficacy=100×(F_{A}−F_{BG})/(F_{C}−F_{BG}),

in which:
F_{A} corresponds to the intracellular level of substrate measured in the cells overexpressing the transporter T (corresponding to the lowest level of fluorescence in the absence of inhibitor),
F_{C} corresponds to the fluorescence in the control cells not expressing the test transporter. This is thus the maximum accumulation of substrate in the cells),
F_{BG} corresponds to the fluorescence measured in the absence of substrate and in the presence of inhibitor.

Cell Growth Test.

The cells are seeded (10 000 cells/well) in 96-well plates and incubated for 24 hours at 37° C. under 5% CO₂. Varied concentrations of mitoxantrone (from 0 to 20 μM in a constant final volume of 100 μL) are then added with an equal volume of each of the test compounds. The cells are then left to divide for 72 hours. The cytotoxicity is evaluated colorimetrically with 3-(4,5-dimethylthiazol-2-yl)-2,5-diphenyltetrazolium bromide (MTT).

A—Efficacy of Inhibition of the Human Efflux Proteins ABC by Azadipeptides

The compounds presented in TABLES 2 to 5 were tested by flow cytometry on mammalian cell lines selectively expressing the efflux proteins ABCG2, ABCB1 or ABCC1, as described previously.

TABLES 6 and 6a show the efficacy of inhibition of the azadipeptides (IA.1), (IA.2), (IA.3), (IB.1) and (IB.11) on the of anticancer drugs by the human ABC pumps.

TABLE 6

| | BCRP/ABCG2 | | | P-gp/ABCB1 | | |
|---|---|---|---|---|---|---|
| | % efficacy | | | % efficacy | | |
| Compound | 10 μM | 2 μM | IC₅₀, μM | 10 μM | 2 μM | IC₅₀, μM |
| (IA.3) | 38 ± 14 | — | — | 98 ± 14 | 90 ± 7 | 0.33 ± 0.09 |
| (IA.1) | 112 ± 6 | 53 ± 10 | 2.0 ± 0.2 | 74 ± 1 | 70 ± 10 | ≦0.5 |
| (IA.2) | 112 ± 26 | 86 ± 14 | 1.06 ± 0.05 | 43 ± 8 | — | — |
| (IB.1) | 87 ± 27 | 72 ± 1 | 0.90 ± 0.05 | 63 ± 5 | 70 ± 20 | 1.0 ± 0.1 |
| (IB.11) | 104 ± 10 | 92 ± 2 | 0.5 ± 0.05 | — | — | 0.7 ± 0.1 |

TABLE 6a

| | MRP1/ABCC1 |
|---|---|
| | % efficacy |
| Compound | 10 μM |
| (IA.3) | — |
| (IA.1) | 14 ± 3 |
| (IA.2) | 5 ± 1 |
| (IB.1) | 5 ± 1 |
| (IB.11) | — |

The mitoxantrone efflux induced by ABCG2 or ABCB1 and the daunorubicin efflux induced by ABCC1 are estimated by flow cytometry, as described previously, at 10 μM of each test compound, and then at 2 μM for the compounds showing at least 80% efficacy at 10 μM. The concentration inducing 50% inhibition (IC₅₀) was estimated for the most efficient aza derivatives. The values are obtained by three independent experiments and were adjusted using the SigmaPlot software.

This test demonstrates the capacity of the products to block the efflux of mitoxantrone (by ABCG2 or ABCB1) or of daunorubicin (by ABCC1).

As shown in TABLE 6, compound (IA.3) is the most efficient for blocking ABCB1 (IC₅₀ of 0.33 μM), whereas its ABCG2 inhibition efficacy is low (38% at 10 μM). The reverse is observed for compound (IA.2), which has an IC₅₀ for ABCG2 of 1 μM and an ABCB1 blocking efficacy of less than 50% at 10 μM. Compound (IA.1) shows better affinity for ABCB1, with an IC₅₀ of the order of 0.5 μM, whereas it is 2 μM for ABCG2. Compounds (IB.1 and IB.11) have the same efficacy on the two transporters, with a micromolar or submicromolar IC₅₀ in both cases. All these compounds appear to have no effect on the efflux protein ABCC1.

B—Compound (IA.2) is a Powerful Noncompetitive Inhibitor of ABCG2

FIG. 2 demonstrates that compound (IA.2) is a noncompetitive inhibitor of ABCG2. The efflux of mitoxantrone by ABCG2 was measured at substrate concentrations ranging from 0 to 20 μM (panels A-C) in the presence or absence of fixed concentrations of compound (IA.2) in a range from 0 to 20 μM (panels D-E). In FIG. 2, the results obtained by varying the inhibitor concentration are presented as a representation, which is either direct in panel A or of Lineweaver-Burke form in panel B or of Eadie-Hofstee form in panel C. The results obtained by varying the substrate concentration are presented in linear (panel D) or logarithmic (panel E) x-axis scale. The curves were adjusted by means of the SigmaPlot software.

The results presented in FIG. 2A by direct representation of the efflux of mitoxantrone as a function of its concentration show that the plateau reached by each curve obtained is different for a given concentration of (IA.2), whereas the maximal half-accumulation concentration of mitoxantrone remains the same irrespective of the inhibitor concentration. These data are characteristic of inhibition of noncompetitive type. This is confirmed by the Lineweaver-Burke and Eadie-Hofstee representations presented, respectively, in FIG. 2B and FIG. 2C. The representations of these two graphs are characteristic of this type of inhibition. The inhibition constant K of compound (IA.2) is estimated as about 1 μM from the adjustment of the points of FIG. 2D or FIG. 2E.

C—Chemosensitization of HEK-293 Cells Expressing ABCG2 by Compound (IA.2).

Increasing concentrations of mitoxantrone (MTX) are applied to HEK-293 cells expressing or not expressing ABCG2 and subjected or not subjected to a treatment with 5 μM of compound (IA.2), and whose residual vitality is evaluated after 72 hours of treatment by an MIT test. The results are illustrated in FIG. 3, in which the cells expressing ABCG2 are represented by circles, and those not expressing it by squares; those that are treated with 5 μM of (IA.2), i.e. five times its IC₅₀, appear as empty symbols, whereas those that are not treated with (IA.2) appear as solid symbols. FIG. 3 shows that incubation of the HEK-293 cells overexpressing ABCG2 with compound (IA.2) restores mitoxantrone sensitivity equal to that observed in the control cells in which ABCG2 is not expressed. Compound (IA.2) thus appears to be a very efficient chemosensitizer.

D—Absence of Cytotoxicity of Compound (IA.2) on the Cell Growth of Human HEK-293 Cells

Increasing concentrations of compound (IA.2) are applied to HEK-293 cells expressing or not expressing ABCG2, and whose residual vitality is evaluated after 72 hours of treatment by an MTT test. The results are illustrated in FIG. 4, in which the cells expressing ABCG2 are represented by empty circles, and those not expressing it by solid circles. The results show that compound (IA.2) is absolutely not cytotoxic for the HEK-293 cells, whether or not they express ABCG2. This absence of toxicity is observed even at a very high concentration such as 40 μM, which corresponds to 40 times the IC₅₀ value.

E—Chemosensitization of HEK-293 Cells Expressing ABCG2 by Compound (IA.1)

Increasing concentrations of mitoxantrone (MTX) are applied to HEK-293 cells expressing or not expressing ABCG2 and subjected or not subjected to a treatment with 5 μM of compound (IA.1), and whose residual vitality is evaluated after 72 hours of treatment by an MTT test. The results are illustrated in FIG. 5, in which the cells expressing ABCG2 are represented by circles, and those not expressing it by squares; those that are treated with 5 μM of (IA.1), i.e. 2.5 times its IC₅₀, appear as empty symbols, whereas those that are not treated with (IA.1) appear as solid symbols. The results show that compound (IA.1) restores virtually full mitoxantrone sensitivity of the cells that overexpress ABCG2, to the same level as that observed in the cells not expressing the transporter. The residual resistance is attributed to the fact that the concentration of product, 5 μM, which corresponds to only 2.5 times the IC₅₀, is thus probably insufficient to impart full sensitization. Compound (IA.1) is thus, itself also, an efficient chemosensitizer.

F—Absence of Cytotoxicity of Compound (IA.1) on Cell Growth

Increasing concentrations of compound (IA.1) are applied to HEK-293 cells expressing or not expressing ABCG2, and whose residual vitality is evaluated after 72 hours of treatment by an MTT test. The results are illustrated in FIG. 6, in which the cells expressing ABCG2 are represented by empty circles, and those not expressing it by solid circles.

The results show that compound (IA.1) has no cytotoxic effect in the concentration range tested, and even at more than 10 times its IC₅₀, whether or not ABCG2 is expressed.

G—Chemosensitization of HEK-293 Cells Expressing ABCG2 by Compound (IB.1)

Increasing concentrations of mitoxantrone (MTX) are applied to HEK-293 cells expressing or not expressing ABCG2 and subjected or not subjected to a treatment with 5 μM of compound (IB.1), and whose residual vitality is evaluated after 72 hours of treatment by an MTT test. The results are illustrated in FIG. 7, in which the cells expressing ABCG2 are represented by circles, and those not expressing it by squares; those that are treated with 5 μM of (IB.1), i.e. 5 times its IC₅₀, appear as empty symbols, whereas those that are not treated with (IB.1) appear as solid symbols. The results show that incubation of the HEK-293 cells overexpressing ABCG2 with compound (IB.1) leads to virtually full restoration of mitoxantrone sensitivity, to a level close to that observed in the cells not expressing the transporter. Compound (IB.1) is thus, itself also, an efficient chemosensitizer.

H—Absence of Cytotoxicity of Compound (IB.1) on Cell Growth

Increasing concentrations of compound (IB.1) are applied to HEK-293 cells expressing or not expressing ABCG2, and whose residual vitality is evaluated after 72 hours of treatment by an MTT test. The results are illustrated in FIG. 8, in which the cells expressing ABCG2 are represented by empty circles and those not expressing it by solid circles. The results show that compound (IB.1) has no cytotoxic effect up to a concentration equivalent to 20 times its IC₅₀, whether or not ABCG2 is expressed. At 40 times the IC₅₀, a relative cytotoxicity appears.

I—Limited Cytotoxicity of Compound (IB.1) on the Growth of NIH-3T3 Cells

Increasing concentrations of compound (IB.1) are applied to NIH-3T3 cells expressing or not expressing ABCB1, and whose residual vitality is evaluated after 72 hours of treatment by an MTT test. The results are illustrated in FIG. 9, in which the cells expressing ABCB1 are represented by empty circles, and those not expressing it by solid circles. The results show that up to 5 times its IC₅₀ (1 μM), the cytotoxicity of compound (IB.1) is negligible. It increases by 30-40% thereafter, but however remains limited to this value, up to a concentration equivalent to 40 times its IC₅₀, whether or not ABCB1 is expressed.

J—Limited Cytotoxicity of Compound (IB.11) on Cell Growth

Increasing concentrations of compound (IB.11) are applied to HEK-293 cells expressing or not expressing ABCG2, and whose residual vitality is evaluated after 72 hours of treatment by an MTT test. The results are illustrated in FIG. 10, in which the cells expressing ABCG2 are represented by empty circles, and those not expressing it by solid circles. The results show that compound (IB.11) has no cytotoxic effect in the concentration range tested, even at more than 80 times its IC₅₀, whether or not ABCG2 is expressed.

K—Compound (IA.3) is a Powerful Noncompetitive Inhibitor of ABCB1

The results presented in FIG. 11 by the direct representation of the efflux of daunorubicin as a function of its concentration, show that the plateau reached by each curve obtained is different for a given concentration of (IA.3), whereas the maximal half-accumulation concentration of daunorubicin remains the same irrespective of the concentration of the inhibitor. These data are characteristic of inhibition of noncompetitive type. This also indicates that this class of inhibitor is generally noncompetitive on ABCB1 and ABCG2 (cf. FIG. 2 and preceding paragraph B).

L—Compound (IA.2) Reduces the Level of Expression of ABCG2, the Protein that it Inhibits

FIG. 12 demonstrates the modulation of the level of expression of ABCG2 by the inhibitor IA.2. HEK293 cells expressing or not expressing BCRP, respectively “HEK293 BCRP” and “HEK293 pcDNA3”, are cultured for the time indicated in the presence of compound IA.2 or DMSO (negative control). They are then harvested and fractionated to analyze their content of ABCG2 and tubulin, the latter being used as a control, as detailed in the Methods section.

The results presented in FIG. 12 show that, in addition to its inhibitory action on ABCG2, compound IA.2 also entails a decrease in the level of expression of the protein, without affecting that of a protein conventionally used as control, tubulin. Compound IA.2 thus makes it possible to modulate not only the activity of ABCG2, but also its level of expression, which proportionately potentiates its action.

1. Compounds, of azapeptide or azapeptidomimetic type, of formula (I): 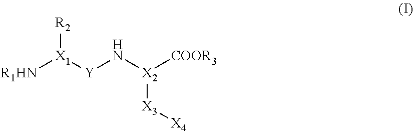
in which:
R₁ represents a protecting group for an amino group, preferably a group —C(O)OR′₁ with R′₁ which represents an alkyl group of 1 to 12 carbon atoms or a group —(CH₂)ₘ₁R″₁ with R″₁ which represents an aryl, cycloalkyl or fluorenyl group and m1 which is equal to 0, 1,2 or 3,
X₁ and X₂ are identical or different and represent —N— or —CH—, it being understood that at least one represents —N—,
R₂ represents a side chain optionally in protected form of an amino acid or an amino acid analog, or an optionally substituted aryl group or an optionally substituted heteroaryl group,
Y represents —CH₂— or —C(O)—,
—X₃-X₄ represents either a group —(CH₂)ₙ—NHR₄ with n equal to 3, 4, 5 or 6, or a group chosen from: 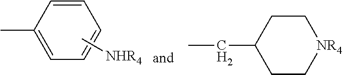
with R₄ which represents a protecting group for an amino group, preferably a group —COOR′₄
with R′₄ which represents an alkyl group of 1 to 12 carbon atoms or a group —(CH₂)ₘ₄R″₄
with R″₄ which represents an aryl or cycloalkyl group and m4 which is equal to 0, 1, 2 or 3,
R₃ represents an alkyl group of 1 to 12 carbon atoms, or a group —(CH₂)ₘ₃R″₃ with R″₃ which represents a cycloalkyl or aryl group, said cycloalkyl and aryl groups possibly being unsubstituted or substituted with one or more groups chosen from: —CH₃, —CF₃, —COOH, —NO₂, —Cl and NH₂ and m3 which is equal to 0, 1, 2 or 3,
in the form of a pure optical isomer or a mixture of optical isomers, and also the salts, solvates or hydrates thereof.

2. Compounds of azapeptide or azapeptidomimetic type as claimed in claim 1, characterized in that R₂ represents a group -L-COOR′₂ with L which represents an aryl group, preferably phenyl, or a chain —(CH₂)m₂— with m₂ which is equal to 1, 2 or 3 and R′₂ which represents an alkyl group of 1 to 12 carbon atoms or a group —(CH₂)^{m′2}R″₂ with R″₂ which represents an aryl group or cycloalkyl, optionally substituted and m′2 which is equal to 0, 1, 2 or 3.

3. Compounds of azapeptide or azapeptidomimetic type as claimed in claim 1, of formula (IA), in the form of a pure isomer or a mixture of isomers, and also the salts, solvates or hydrates thereof: 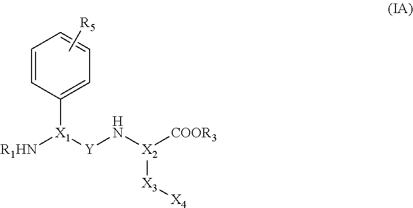
in which R₅ represents a group —NH₂, —NO₂, —OH, —O-alkyl of 1 to 8 carbon atoms, O-(CH₂)ₘ₅R′₅ with R′₅ which represents an aryl group and m5 which is equal to 0 or 1 or alternatively R₅ represents a group —COOR″₅ with R″₅ which represents an alkyl group of 1 to 12 carbon atoms or a group —(CH₂)_{m′5}R′″₅ with R′″₅ which represents an aryl or cycloalkyl group and m′5 which is equal to 0, 1, 2 or 3, R₁, X₁, Y, X₂, X₃, X₄ and R₃ being as defined for (I) in claim 1.

4. Compounds of azapeptide or azapeptidomimetic type of formula (IA) as claimed in claim 3, characterized in that R₅ represents a group —COOR″₅ with R″₅ which represents an ethyl, benzyl or —CH[CH(CH₃)₂]₂ group.

5. Compounds of azapeptide or azapeptidomimetic type of formula (IA) as claimed in claim 3, characterized in that the group R₅ is in the meta position.

6. Compounds of azapeptide or azapeptidomimetic type of formula (IA) as claimed in claim 3, characterized in that the group R₅ is in the para position.

7. Compounds of azapeptide or azapeptidomimetic type of formula (IA) as claimed in claim 3, characterized in that X₁ represents —N— and X₂ represents —CH—.

8. Compounds of azapeptide or azapeptidomimetic type of formula (IA) as claimed in claim 3, characterized in that X₁ and X₂ represent N.

9. Compounds of azapeptide or azapeptidomimetic type of formula (IA) as claimed in claim 3, characterized in that Y represents —C(O)—.

10. Compounds of azapeptide or azapeptidomimetic type of formula (IA) as claimed in claim 3, characterized in that —X₃-X₄ represents a group —(CH₂)ₙ—NHR₄ with n equal to 4 or 6 and R₄ as defined for said formula (I).

11. Compounds of azapeptide or azapeptidomimetic type as claimed in claim 1 of formula (IB), in the form of a pure isomer or a mixture of isomers, and also the salts, solvates or hydrates thereof: 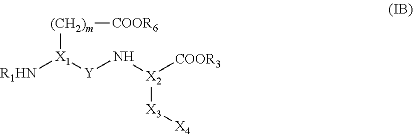
in which m is equal to 1, 2 or 3, R₆ represents an alkyl group of 1 to 12 carbon atoms, or a group —(CH₂)ₘ₆R′₆ with R′₆ which represents a cycloalkyl or aryl group, said cycloalkyl and aryl groups possibly being unsubstituted or substituted with one or more groups chosen from: —CH₃, —CF₃, —COOH, —NO₂, —Cl and NH₂ and m6 which is equal to 0, 1, 2 or 3, R₁, X₁, Y, X₂, X₃, X₄ and R₃ being as defined for (I) in claim 1.

12. Compounds of azapeptide or azapeptidomimetic type of formula (IB) as claimed in claim 11, characterized in that m is equal to 1 or 2.

13. Compounds of azapeptide or azapeptidomimetic type of formula (IB) as claimed in claim 11, characterized in that R₆ represents an ethyl, benzyl, or —CH[CH(CH₃)₂]₂ group.

14. Compounds of azapeptide or azapeptidomimetic type of formula (IB) as claimed in claim 11, characterized in that X₁ represents —CH—.

15. Compounds of azapeptide or azapeptidomimetic type of formula (IB) as claimed in claim 11, characterized in that X₂ represents —N—.

16. Compounds of azapeptide or azapeptidomimetic type of formula (IB) as claimed in claim 11, characterized in that —X₃-X₄ represents a group —(CH₂)ₙ—NHR₄ with n equal to 6 and R₄ as defined for said formula (I).

17. Compounds of azapeptide or azapeptidomimetic type of formula (I) as claimed in claim 1, characterized in that R₁ represents a group —COOR′₁ and R′₁ represents a tert-butyl or benzyl group.

18. Compounds of azapeptide or azapeptidomimetic type of formula (I) as claimed in claim 1, characterized in that R₃ represents a tert-butyl or benzyl group.

19. Compounds of azapeptide or azapeptidomimetic type of formula (I) as claimed in claim 1, characterized in that R₄ represents —COOR′₄ with R′₄ which represents a benzyl or tert-butyl group.

20. Compounds of azapeptide or azapeptidomimetic type as claimed in claim 1, chosen from:
tert-butyl ester of (2S)-6-(benzyloxycarbonylamino)-2-[1-(2′-(benzyloxycarbonyl)phenyl)-2-(tert-butoxycarbonyl)hydrazinocarbonylamino]hexanoic acid (IA.1)
tert-butyl ester of (2S)-6-(benzyloxycarbonylamino)-2-[1-(3′-(benzyloxycarbonyl)phenyl)-2-(tert-butoxycarbonyl)hydrazinocarbonylamino]hexanoic acid (IA.2)
tert-butyl ester of (2S)-6-(benzyloxycarbonylamino)-2-[1-(4′-(benzyloxycarbonyl)phenyl)-2-(tert-butoxycarbonyl)hydrazinocarbonylamino]hexanoic acid (IA.3)
benzyl ester of (2R)-6-(benzyloxycarbonylamino)-2-[1-(3′-(benzyloxycarbonyl)phenyl)-2-(tert-butoxycarbonyl)hydrazinocarbonylamino]hexanoic acid (IA.4)
benzyl ester of (2S)-6-(benzyloxycarbonylamino)-2-[1-(3′-(benzyloxycarbonyl)phenyl)-2-(tert-butoxycarbonyl)hydrazinocarbonylamino]hexanoic acid (IA.5)
tert-butyl ester of (2S)-6-(benzyloxycarbonylamino)-2-[1-(3′-(phenyloxycarbonyl)phenyl)-2-(tert-butoxycarbonyl)hydrazinocarbonylamino]hexanoic acid (IA.6)
benzyl ester of 2-[1-(3′-(benzyloxycarbonyl)phenyl)-2-(tert-butoxycarbonyl)hydrazinocarbonyl]-1-(6-benzyloxycarbonylaminohexyl)hydrazinocarboxylic acid (IA.7)
benzyl ester of 2-[1-(3′-(benzyloxycarbonyl)phenyl)-2-(tert-butoxycarbonyl)hydrazinocarbonyl]-1-(6-butyloxycarbonylaminohexyl)-hydrazinocarboxylic acid (IA.8)
benzyl ester of (3S)-4-[2-(6-benzyloxycarbonylaminohexyl)-2-tert-butoxycarbonylhydrazino]-3-tert-butoxycarbonylaminobutyric acid (IB.1)
benzyl ester of (3S)-4-[2-(3-benzyloxycarbonylaminopropyl)-2-tert-butoxycarbonylhydrazino]-3-tert-butoxycarbonylamino-4-oxobutyric acid (IB.2)
benzyl ester of (3S)-4-[2-(4-benzyloxycarbonylaminophenyl)-2-tert-butoxycarbonylhydrazino]-3-tert-butoxycarbonylamino-4-oxobutyric acid (IB.3)
benzyl ester of (4S)-5-[2-(4-benzyloxycarbonylaminophenyl)-2-tert-butoxycarbonylhydrazino]-4-tert-butoxycarbonylamino-5-oxopentanoic acid (IB.4)
benzyl ester of (3S)-4-[2-(5-benzyloxycarbonylaminopentyl)-2-tert-butoxycarbonylhydrazino]-3-tert-butoxycarbonylamino-4-oxobutyric acid (IB.5)
benzyl ester of 4-[2((2S)-3-benzyloxycarbonyl-2-tert-butoxycarbonylaminopropyl)-1-tert-butoxycarbonylhydrazinomethyl]piperidine-1-carboxylic acid (IB.6)
benzyl ester of 4-[2-((2S)-3-benzyloxycarbonyl-2-tert-butoxycarbonylaminopropionyl)-1-tert-butoxycarbonylhydrazinomethyl]piperidine-1-carboxylic acid (IB.7)
benzyl ester of (3S)-4-[2-(5-benzyloxycarbonylaminopentyl)-2-tert-butoxycarbonylhydrazino]-3-tert-butoxycarbonylaminobutyric acid (IB.8)
benzyl ester of (3S)-4-[2-(6-benzyloxycarbonylaminohexyl)-2-tert-butoxycarbonylhydrazino]-3-tert-butoxycarbonylamino-4-oxobutyric acid (IB.9)
benzyl ester of (3S)-3-tert-butoxycarbonylamino-4-[2-tert-butoxycarbonyl-2-(6-tert-butoxycarbonyl-aminohexyl)hydrazino]butyric acid (IB.10)
benzyl ester of (3S)-4-[2-benzyloxycarbonyl-2-(6-benzyloxycarbonylaminohexyl)hydrazino1-3-tert-butoxycarbonylaminobutyric acid (IB.11)
benzyl ester of (3S)-3-benzyloxycarbonylamino-4-[2-benzyloxycarbonyl-2-(6-benzyloxycarbonyl-aminohexyl)hydrazino]butyric acid (IB.12)
benzyl ester of (3S)-4-[2-benzyloxycarbonyl-2-(6-tert-butoxycarbonylaminohexyl)hydrazino]-3-tert-butoxycarbonylaminobutyric acid (IB.13)
tert-butyl ester of (2S)-6-(benzyloxycarbonylamino)-2-[1-(benzyloxycarbonylmethyl)-2-(tert-butoxycarbonyl)hydrazinocarbonylamino]hexanoic acid (IB.14),
and also salts, solvates and/or hydrates thereof, and especially the pharmaceutically acceptable forms thereof.

21. The compound of azapeptide or azapeptidomimetic type as claimed in claim 1, as an adjuvant for a therapeutic treatment administered to a patient, for which the body of the treated patient develops a resistance, which is intended to restore the activity of the therapeutic treatment.

22. The compound of azapeptide or azapeptidomimetic type as claimed in claim 1, as an adjuvant for an anticancer or anti-infectious medicament.

23. The compound of azapeptide or azapeptidomimetic type as claimed in claim 1, as an adjuvant for a medicament chosen from anthracyclines, topoisomerase inhibitors, antimetabolic agents (antifolates), tyrosine kinase inhibitors, antiviral agents (reverse transcriptase inhibitors) and antiparasitic agents.

24. The compound of azapeptide or azapeptidomimetic type as claimed in claim 1, as an adjuvant for a medicament chosen from daunorubicin, doxorubicin, mitoxantrone, camptothecin and derivatives thereof, irinotecan, topotecan, indolocarbazoles, methotrexate, imatinib, gefitinib, zidovudine, lamivudine, abacavir, ivermectin, albendazole, oxfendazole and ketoconazole.

25. Compounds of azapeptide or azapeptidomimetic type of formula (IA) as claimed in claim 3, characterized in that either: (1) R₁ represents a group —COOR′₁ and R′₁ represents a tert-butyl or benzyl group; (2) R₃ represents a tert-butyl or benzyl group; or (3) R₄ represents —COOR′₄ with R′₄ which represents a benzyl or tert-butyl group.

26. Compounds of azapeptide or azapeptidomimetic type of formula (IB) as claimed in claim 11, characterized in that either: (1) R₁ represents a group —COOR′₁ and R′₁ represents a tert-butyl or benzyl group; (2) R₃ represents a tert-butyl or benzyl group; or (3) R₄ represents —COOR′₄ with R′₄ which represents a benzyl or tert-butyl group.
